(19) United States
(12) Patent Application Publication
(10) Pub. No.: US 2019/0201414 A1
(43) Pub. Date: Jul. 4, 2019

(54) INHIBITOR OF HEME DEGRADATION FOR USE TO IMPROVE ANTIBIOTIC TREATMENT OF MYCOBACTERIUM TUBERCULOSIS INFECTION

(71) Applicant: The United States of America, as represented by the Secretary, Department of Health and Human Serv, Bethesda, MD (US)

(72) Inventors: Diego Luis Costa, Rockville, MD (US); Bruno Bezerril Andrade, Salvador (BR); Franklin Alan Sher, Potomac, MD (US)

(73) Assignee: The United States of America, as represented by the Secretary, Department of Health and Human Serv, Bethesda, MD (US)

(21) Appl. No.: 16/311,876

(22) PCT Filed: Jun. 29, 2017
(86) PCT No.: PCT/US2017/039935

§ 371 Date: Dec. 20, 2018

Related U.S. Application Data
(60) Provisional application No. 62/357,558, filed on Jul. 1, 2016.

(51) Int. Cl. A61K 31/555; A61K 31/4409; A61K 31/4965; A61K 31/496; A61P 31/06

(52) U.S. Cl. CPC A61K 31/555; A61K 31/4409; A61P 31/06; A61K 31/496; A61K 31/4965

(57) ABSTRACT

The present invention provides a method of preventing or treating a Mycobacterium tuberculosis (Mtb) infection in a mammal the method comprising administering to the mammal a first inhibitor, wherein the first inhibitor is an inhibitor of heme degradation and wherein the first inhibitor is a metal protoporphyrin, and administering to the mammal a second inhibitor, wherein the second inhibitor is an inhibitor of Mtb, wherein administration of the first and second inhibitors to the mammal prevents or treats Mtb infection in the mammal.

### PRIORITY INFORMATION

This application claims the benefit of U.S. Provisional Application No. 62/357,558, filed Jul. 1, 2016. The disclosure of this provisional application is incorporated herein in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,664 Byte ASCII (Text) file named “729024_ST25.txt,” dated Jun. 26, 2017.

### BACKGROUND OF THE INVENTION

The World Health Organization estimates that in 2014 there were 9.6 million cases of Mycobacterium tuberculosis infection, of which 1.5 million cases led to death. Of these cases, a vast majority occurred in the developing world. There is no effective vaccine against tuberculosis, and the standard antibiotic treatment takes at least 6 months. Due to the long length of treatment, many patients fail to adhere to the standard chemotherapy regimen, which can lead to disease reactivation and in some cases bacterial resistance to the standard drugs. This has promoted the emergence of multi-drug resistant bacterial strains, highlighting the need for new treatments.

### BRIEF SUMMARY OF THE INVENTION

The present invention provides a method of preventing or treating a Mycobacterium tuberculosis (Mtb) infection in a mammal, the method comprising administering to the mammal a first inhibitor, wherein the first inhibitor is an inhibitor of heme degradation and wherein the first inhibitor is a metal protoporphyrin, and administering to the mammal a second inhibitor, wherein the second inhibitor is an inhibitor of Mtb, wherein administration of the first and second inhibitors to the mammal prevents or treats Mtb infection in the mammal.

### BRIEF DESCRIPTION OF THE DRAWINGS

FIG. 1 presents a diagram of the pathway of heme degradation by heme oxygenase-1 (HO-1).

FIG. 2 presents the chemical structures of inhibitors in accordance with embodiments of the invention.

FIG. 3 presents two dot plots which show use of an inhibitor of heme degradation enhances host resistance to M. tuberculosis in accordance with embodiments of the invention. Dot plots show individual log 10 CFU values and means (*p<0.05; ***p<0.001; n.s.=non-significant).

FIG. 4 presents two dot plots which show use of an inhibitor of heme degradation enhances host resistance to M. tuberculosis in accordance with embodiments of the invention. Dot plots show individual log 10 CFU values (*p<0.05; ***p<0.001; n.s.=non-significant). Each experimental group consisted of 4 to 5 mice. Each panel shows the results of a representative experiment of 2 to 4 performed. Left panel, mice were euthanized at 3 weeks post treatment (wpi). Right panel, mice were euthanized 6 wpi.

FIG. 5 presents three dot plots which show use of an inhibitor of heme degradation enhances host resistance to M. tuberculosis in accordance with embodiments of the invention. Dot plots show individual log 10 CFU values and means (*p<0.05; ***p<0.001; n.s.=non-significant). Mice were euthanized 3 wpi.

FIG. 6 presents two dot plots which show use of an inhibitor of heme degradation enhances host resistance to M. tuberculosis in wild type mice (WT), but has no effect in T-cell receptor α-deficient mice (TCR-α^{-/-}) in accordance with embodiments of the invention. Dot plots show individual log 10 CFU values and means (*p<0.05; ***p<0.001; n.s.=non-significant).

FIG. 7 presents two dot plots which show use of an inhibitor of heme degradation enhances host resistance to M. tuberculosis in wild type mice (WT), but has no effect in T-cell receptor α-deficient mice (TCR-α^{-/-}) in accordance with embodiments of the invention. Dot plots show individual log 10 CFU values and means (*p<0.05; ***p<0.001; n.s.=non-significant).

FIG. 8 presents two graphs which show heme degradation (left panel) and SnPPIX degradation (right panel) by MhuD. Each line represents a different time point. The Y axis presents the absorbance value, and the X axis presents the Wavelength in nm.

FIG. 9 presents a graph which shows heme degradation by MhuD in the presence of SnPPIX. Each line represents a different time point. The Y axis presents the Δabsorbance value ([absorbance of 5 μM MhuD-heme+2 μM SnPPIX]−[absorbance of 2 μM SnPPIX]), and the X axis presents the Wavelength in nm.

FIG. 10 presents two graphs showing heme degradation by recombinant human HO-1-G139A (hHO-1) in the absence and presence of SnPPIX. Each line represents a different time point. The Y axis on the left panel presents the absorbance, and the Y axis on the right panel presents the Δabsorbance value ([absorbance of 5 μM MhuD-heme+2 μM SnPPIX]−[absorbance of 2 μM SnPPIX]). In both panels the X axis presents the wavelength in nm.

FIG. 11 presents a graph of the relative HO-1 mRNA expression, measured by real time PCR in lungs of M. tuberculosis-infected C57BL/6 mice (WT) and TCR-α^{-/-} mice at 1, 2, 3, 4, and 5 wpi. The dotted line in the graph, represents the basal expression of that gene in lungs of uninfected animals.

FIG. 12 presents A graph of the M. tuberculosis log 10 CFU values in the lungs of infected WT and TCR-α^{-/-} mice assayed at day 1 (0 wpi) and 1, 2, 3, 4, and 5 wpi. Graph shows means±standard deviation of results. *p<0.05.

FIG. 13 presents graphs of MhuD mRNA expression in lungs of C57BL/6 (WT) and TCR-α^{-/-} mice at 4 and 5 wpi. Results are expressed as mean femtograms/ml of cDNA per bacteria in each sample±standard deviation (left panel) and as the ratio between the average MhuD gene expression in WT and TCR-α^{-/-} mouse lung samples (right panel).

FIG. 14 presents two dot plots which show use of an inhibitor of heme degradation enhances host resistance to M. tuberculosis in wild type mice (WT), but has no effect in T-cell receptor α-deficient mice (TCR-α^{-/-}) in accordance with embodiments of the invention. Dot plots show individual log 10 CFU values and means (*p<0.05; ***p<0.001; n.s.=non-significant).

FIG. 15 presents a plot of a time course of the quantification of CFU in lungs of an M. tuberculosis infected WT mouse in the presence and absence of an inhibitor of heme degradation. The X axis presents the weeks post infection and the Y axis presents the log 10 CFU. The dotted line represents the limit of detection of the assay.

FIG. 16 presents a plot of a time course of the quantification of CFU in lungs of M. tuberculosis infected WT mice in the presence and absence of an inhibitor of heme degradation. The X axis presents the weeks post infection and the Y axis presents the log 10 CFU. The dotted line represents the limit of detection of the assay.

FIG. 17 presents a graph of the ratio of the mean HO-1 mRNA expression in lungs of RHZ treated vs untreated Mtb-infected mice at 3, 6, and 9 weeks post treatment initiation (wpt).

FIG. 18 presents graphs of IFN-γ expression in CD4+ T lymphocytes in lung homogenates of untreated or RHZ treated Mtb-infected mice at the indicated time points after initiation of therapy.

FIG. 19 presents graphs of the The IFN-γ expression in CD8+ T lymphocytes in lung homogenates of untreated or RHZ treated Mtb-infected mice at the indicated time points after initiation of therapy.

### DETAILED DESCRIPTION OF THE INVENTION

The present invention provides a method of preventing or treating an Mtb infection in a mammal, the method comprising administering to the mammal a first inhibitor, wherein the first inhibitor is an inhibitor of heme degradation and wherein the first inhibitor is a metal protoporphyrin, and administering to the mammal a second inhibitor, wherein the second inhibitor is an inhibitor of Mtb, wherein administration of the first and second inhibitors to the mammal prevents or treats Mtb infection in the mammal.

As shown in the Example below, use of an inhibitor of heme degradation markedly enhances bacterial clearance in Mtb-infected mice undergoing conventional antibiotic therapy—and does so without obvious toxic side effects to the host.

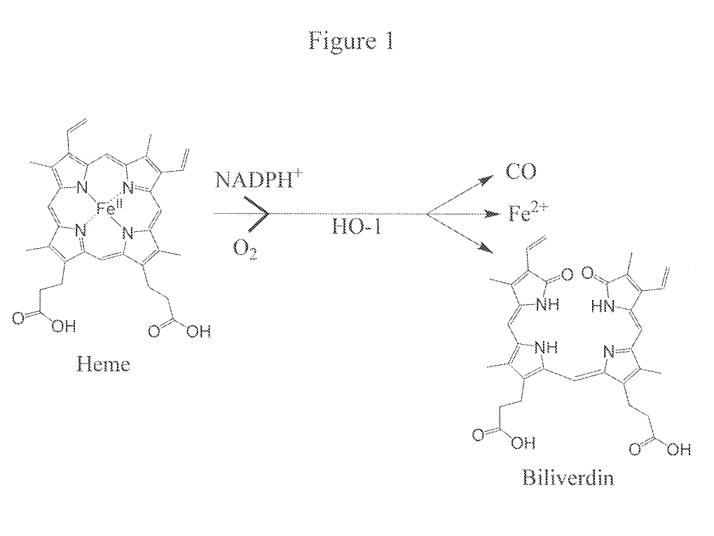

Without wishing to be bound by theory, the first inhibitor may be an inhibitor of host heme oxygenase-1 (HO-1). Host HO-1 expression is induced during Mycobacterium tuberculosis (Mtb) infection; individuals presenting more severe forms of disease express higher levels of the enzyme; and HO-1 returns to baseline levels following successful treatment of the infection. HO-1 is a cytoprotective enzyme with anti-oxidant properties and is also induced in response to oxidative stress. Its activity results in the cleavage of free heme, releasing carbon monoxide, biliverdin and ferrous iron (FIG. 1). Iron is an important nutrient for Mtb. Thus, host HO-1 may be utilized by the pathogen to promote the pathogen's own survival. Targeting host factors that are involved during the infectious process is not expected to result in development of resistant bacteria since, in contrast to antibiotics, the pathogens themselves are not targeted. This host-directed strategy may have an added advantage as a treatment for infections with already antibiotic-resistant Mtb strains.

Without wishing to be bound by theory, the substrate-binding site of HO-1 recognizes the side chain of the porphyrin ring but not the metal ion in its center; because of this, other metal protoporphyrins, e.g., ZnPPIX and SnPPIX, are able to bind HO-1. In contrast to iron, tin and zinc ions do not bind molecular oxygen; due to this ZnPPIX and SnPPIX, for example, cannot be degraded by HO-1 but inhibit the activity of HO-1 through competition with its natural heme substrate. SnPPIX presents a well-known potent HO-1 inhibitor activity and has been extensively used for this purpose experimentally. SnPPIX exhibits higher heme oxygenase inhibitory capacity compared with ZnPPIX, and SnPPIX has been used clinically as to treat hiperbilirrubinemia in newborns with minimum side effects. Tin mesoporphyrin may be more potent than SnPPIX in its heme oxygenase inhibitory capacity. The choice of the inhibitor can be based host toxicity and potency of HO-1 inhibition. The choice of inhibitor also can be based on other criteria, such as solubility of the inhibitor. For example, ZnPPIX is less soluble than SnPPIX, and use of ZnPPIX may require solubilizing agents.

Without wishing to be bound by theory, the first inhibitor may be an inhibitor of a bacterial enzyme that catalyzes heme degradation, MhuD, a bacterial homolog of heme oxygenase. MhuD differs from mammal HO-1 structurally and in its mode of action, and heme binding to MhuD is distinct from that of HO-1: up to two heme molecules can be bound at the same time at the MhuD active site. Also, heme degradation by MhuD results in the release of biliverdin and iron but does not generate carbon monoxide. MhuD may bind metalloporphyrin inhibitors, which could promote bacterial clearance by inhibiting MhuD.

In an embodiment, the first inhibitor, which is a metal protoporphyrin, is tin protoporphyrin IX. In another embodiment, the first inhibitor is zinc protoporphyrin IX. In another embodiment, the first inhibitor is gallium protoporphyrin IX. In another embodiment, the first inhibitor is any one of tin mesoporphyrin IX; zinc deuteroporphyrin IX 2,3, bisethyleneglycol; chromium protoporphyrin IX; or chromium mesoporphyrin IX.

In an embodiment, the first inhibitor is tin (IV) protoporphyrin IX dichloride (SnPPIX). In another embodiment, the first inhibitor is zinc (II) protoporphyrin IX (ZnPPIX). In another embodiment, the first inhibitor is gallium (III) protoporphyrin IX chloride (GaPPIX). In another embodiment, the first inhibitor is any one of tin (IV) mesoporphyrin IX dichloride (SnMPIX); zinc (II) deuteroporphyrin IX 2,3, bisethyleneglycol (ZnBG); chromium (III) protoporphyrin IX choride (CrPPIX); or chromium (III) mesoporphyrin IX choride (CrMPIX).

In an embodiment, any combination of the above first inhibitors may be used.

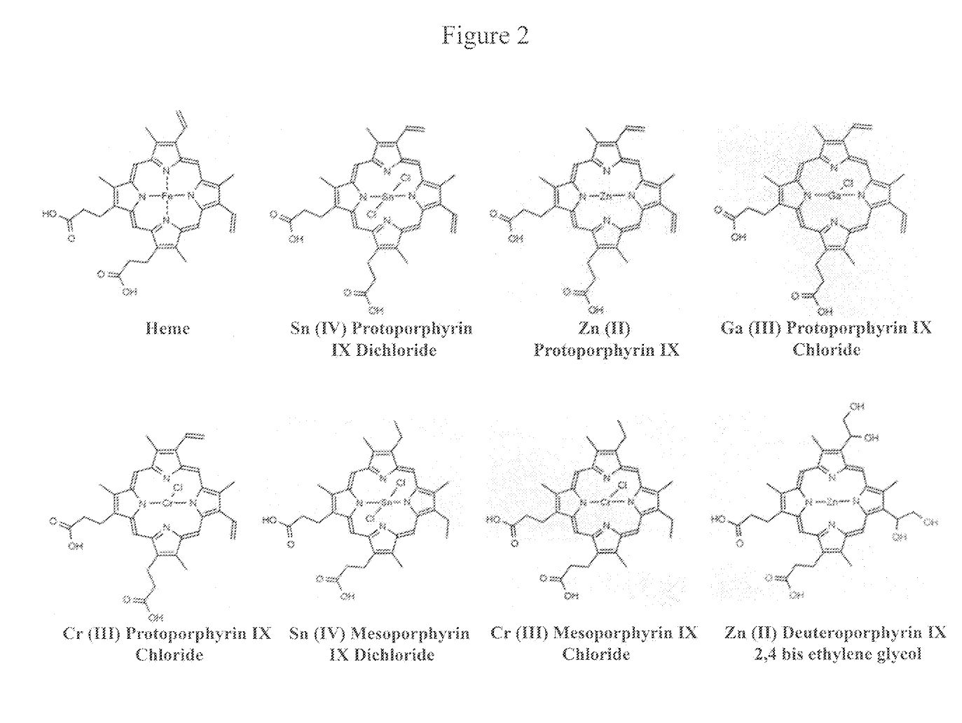

FIG. 2 shows the structures of heme, tin (IV) protoporphyrin IX dichloride, zinc (II) protoporphyrin IX, gallium (III) protoporphyrin IX chloride, chromium (III) protoporphyrin IX choride, tin (IV) mesoporphyrin IX dichloride, chromium (III) mesoporphyrin IX choride, and zinc (II) deuteroporphyrin IX 2,3, bisethyleneglycol.

In an embodiment, the second inhibitor, which is an inhibitor of Mtb, is one or more of isoniazid, rifampicin, pyrazinamide, ethambutol, streptomycin, rifabutin, kanamycin, amikacin, capreomycin, levofloxacin, moxifloxacin, ofloxacin, para-aminosalicylic acid, cycloserine, terizidone, thionamide, protionamide, clofazimine, linezolid, amoxicilin/clavulonate, thiocetazone, lmipenem/cilastatin, sutezolid, clarithromycin, bedaquiline, pretomanid, and TBA-354. Other inhibitors include those in U.S. Pat. No. 8,450,368, which is incorporated by reference herein in its entirety. In an embodiment, the inhibitor of Mtb is pyrazinamide, rifampicin, isoniazid, or any combination thereof. One or more conventional inhibitors of Mtb may be used as the second inhibitor.

In the following compounds, any atom (e.g., N or O) that is not shown in its full valency is understood to complete its valency with H.

In another embodiment, the second inhibitor is a compound of General Formula I:

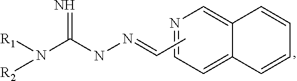

where R₁ and R₂ are independently hydrogen or

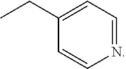

In another embodiment, the second inhibitor is a compound of General Formula II:

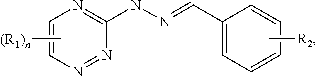

where n is 1 or 2, R₁ and R₂ are independently aryl, halogen, Cl,

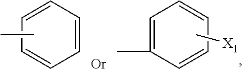

where X₁ is halogen or Cl, or when n is 2, two R₁ groups may form a heteroaryl ring.

In another embodiment, the second inhibitor is a compound of General Formula III:

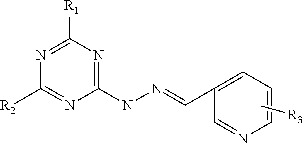

where R₁ and R₂ are

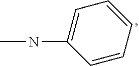

and R₃ is halogen or Cl.

In another embodiment, the second inhibitor is a compound of General Formula IV:

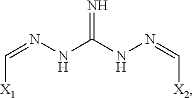

where X₁ and X₂ are

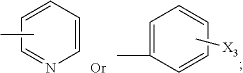

where X₃ is halogen, Br or F.

In another embodiment, the second inhibitor is a compound of General Formula V:

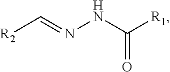

where one of R₁ or R₂ is

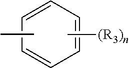

where n is 0-5, R₃ is halogen, Cl, Br, F, or two R₃s together form a naphthyl ring and the other is

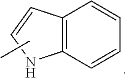

Preferably, one of R₁ or R₂ is

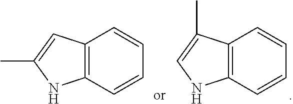

In another embodiment, the second inhibitor is a compound of General Formula VI:

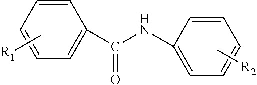

wherein R₁ and R₂ are independently H, N(NH), NH₂, OCOCH₃, COO—, COOH,

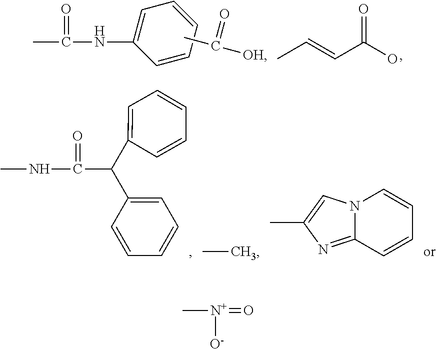

In another embodiment, the second inhibitor is a compound of General Formula VII:

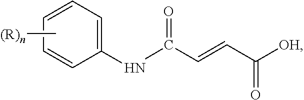

where n is 1 or 2, R is —NH-phenyl,

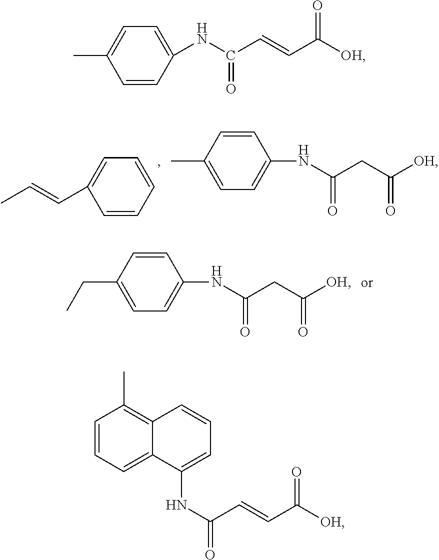

or where n is 2 and the two Rs form a naphthyl ring.

In another embodiment, the second inhibitor is a compound of General Formula VIII:

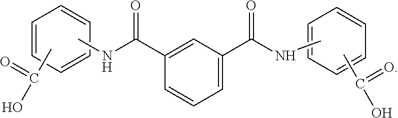

In another embodiment, the second inhibitor is a compound of General Formula IX:

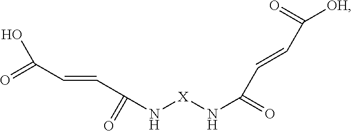

where X is

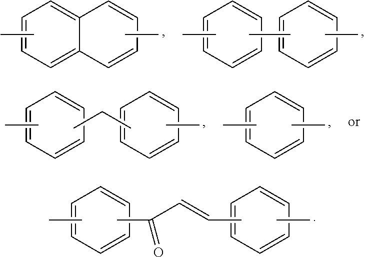

In another embodiment, the second inhibitor is a compound of General Formula X:

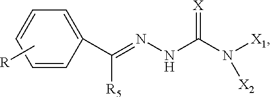

where X is S, O, N(NH), X¹ and X² are independently halogen, Cl, methyl, H or

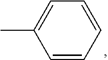

### R is

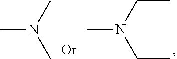

### R₅ is H or

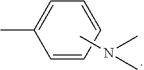

Preferably R is in the para position.

In another embodiment, the second inhibitor is a compound of General Formula XI:

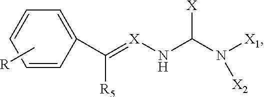

where X is SH or

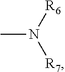

where R₆ and R₇ are independently H, methyl, phenyl, or benzyl, and R, R₅, X₁ and X₂ are as shown in General Formula X.

In another embodiment, the second inhibitor is a compound of General Formula XII:

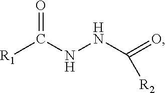

where R₁ and R₂ are independently methyl, heteroaryl, aryl, or any one of the following:

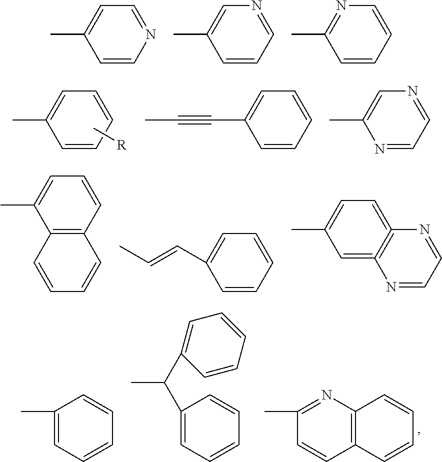

where R is selected from C₁ to C₆ alkyl, including methyl.

In another embodiment, the second inhibitor is a compound of General Formula XIII:

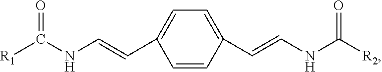

wherein R1 and R2 are the same in General Formula XII.

In another embodiment, the second inhibitor is a compound of General Formula XIV:

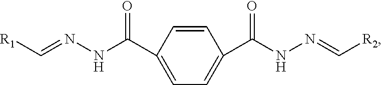

wherein R1 and R2 are the same in General Formula XII.

In another embodiment, the second inhibitor is one of the following compounds:

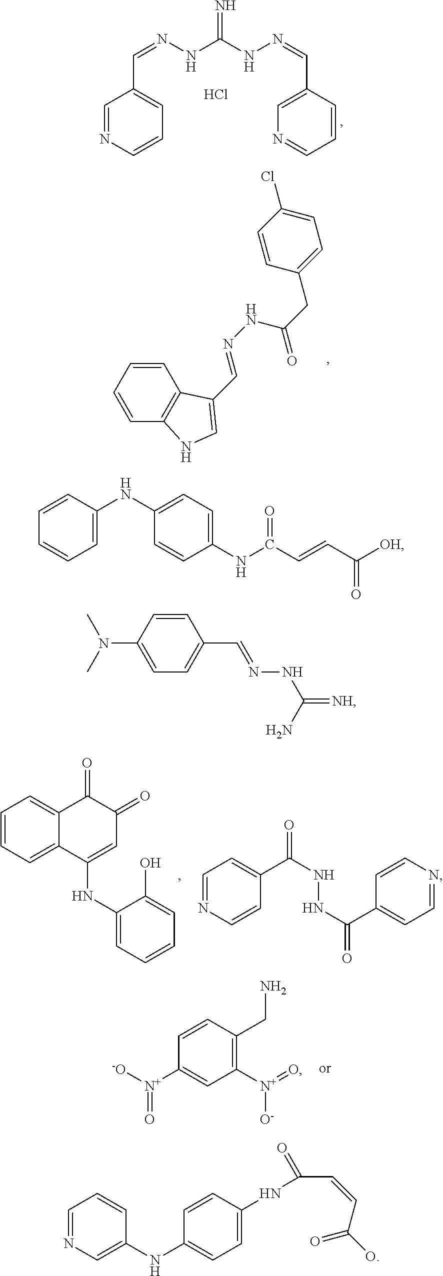

In another embodiment, the second inhibitor is one of the following compounds:

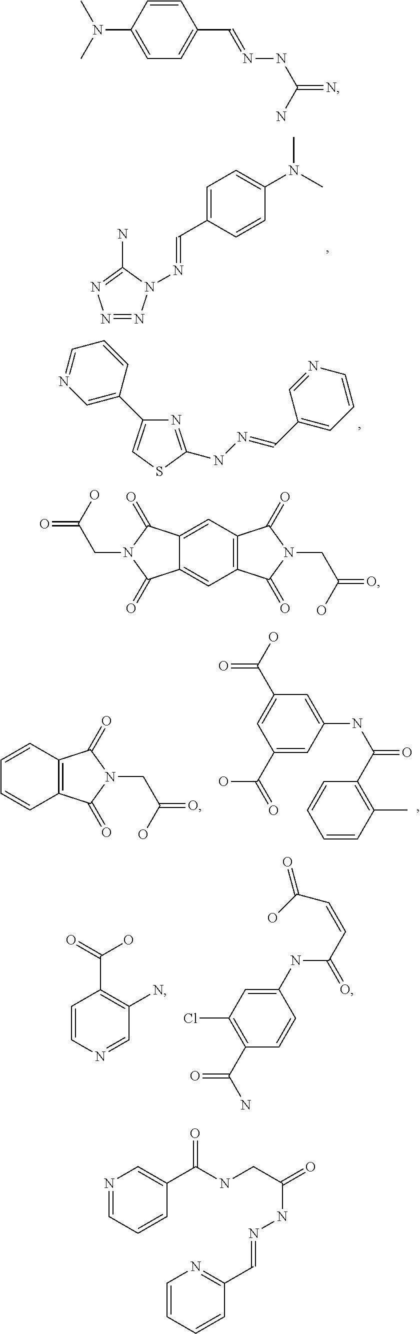

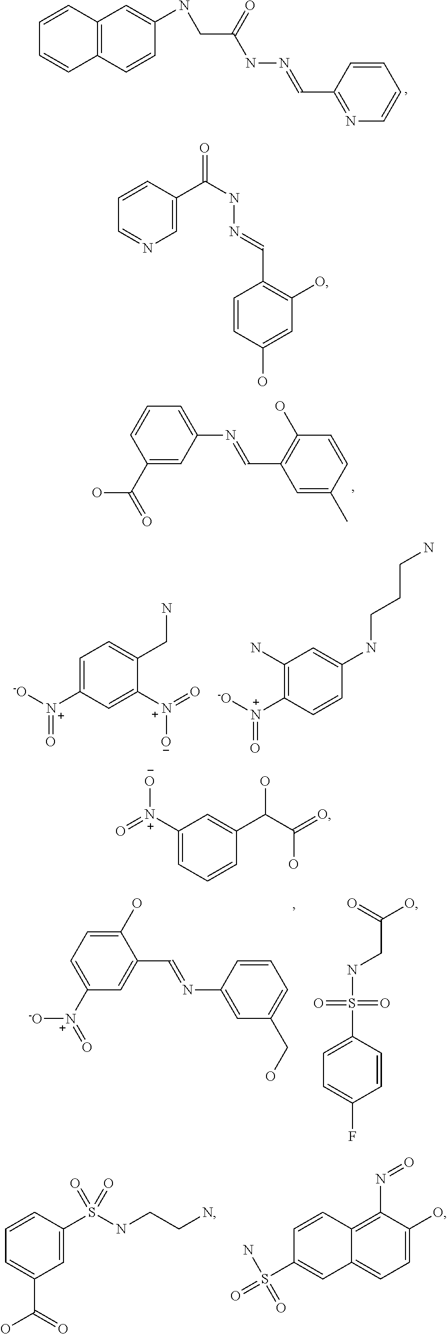

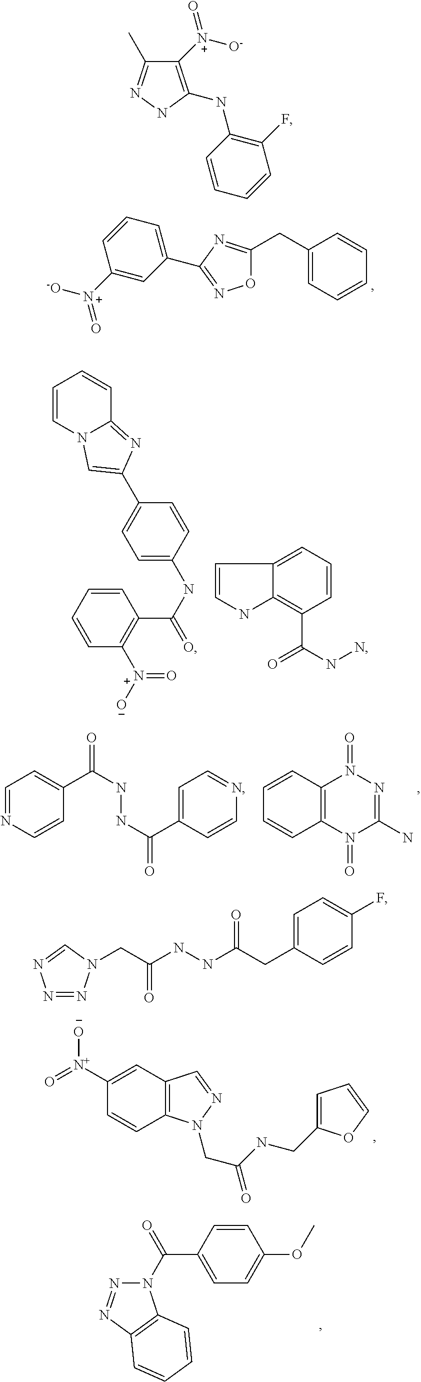

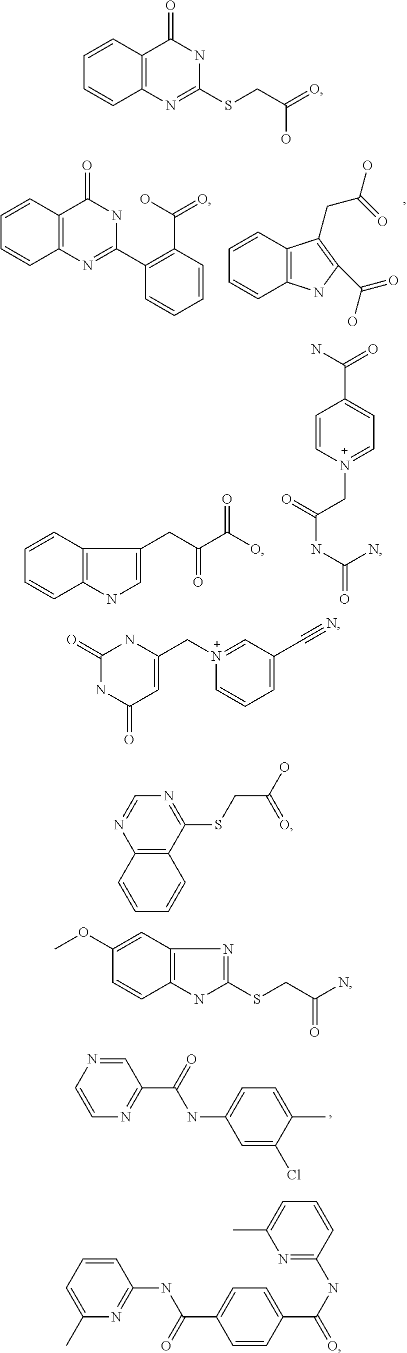

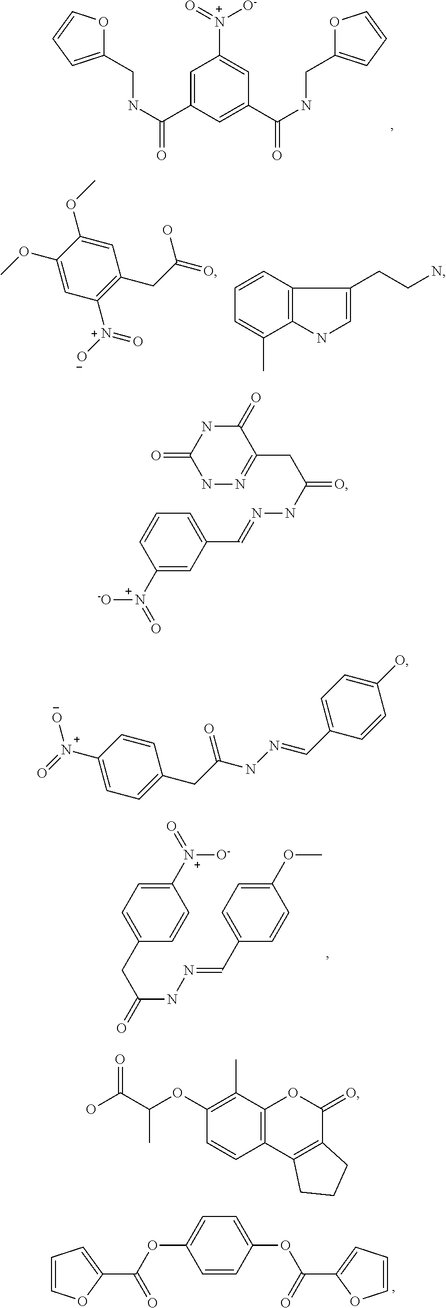

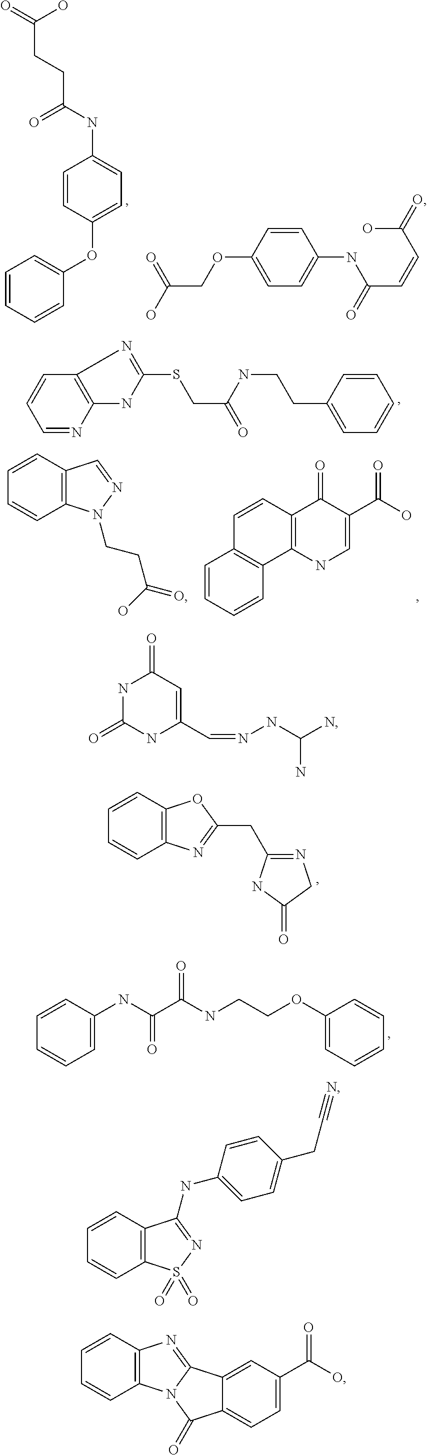

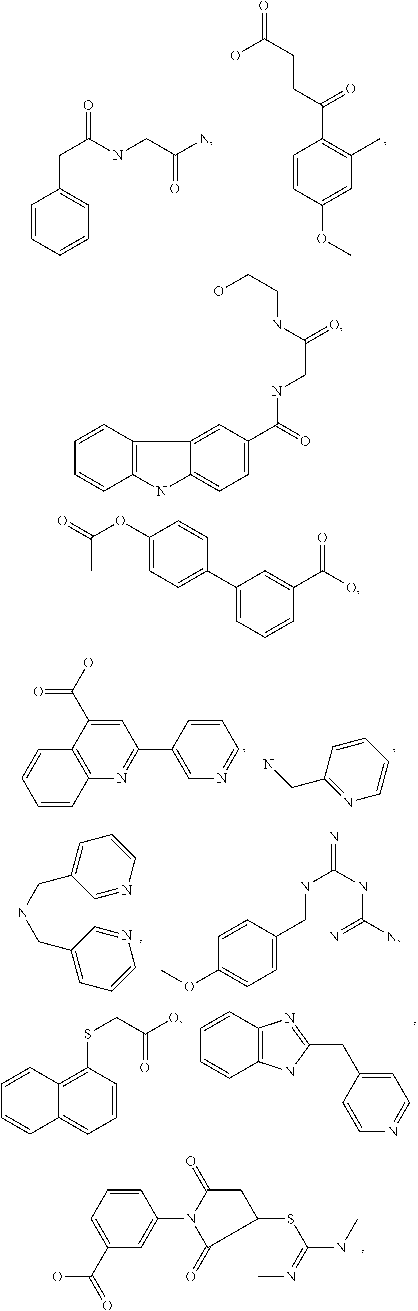

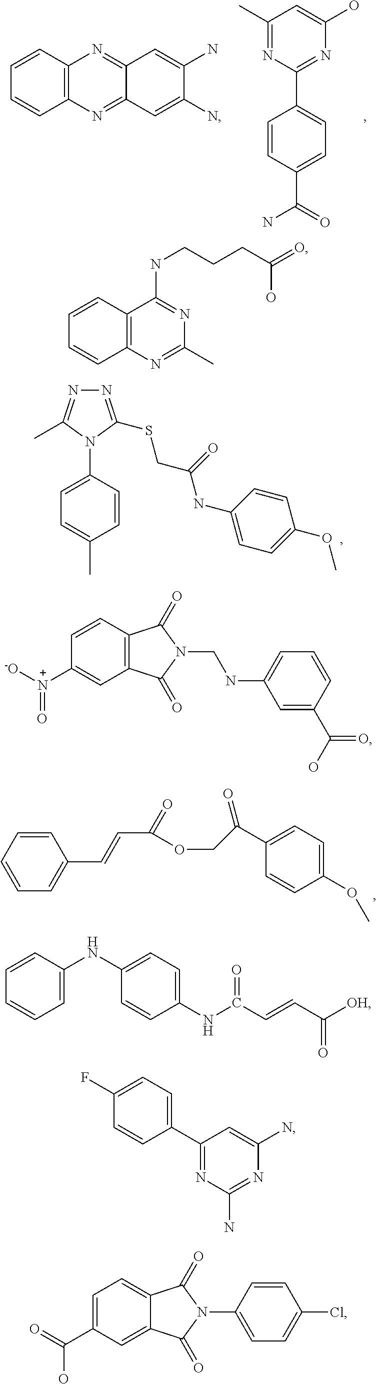

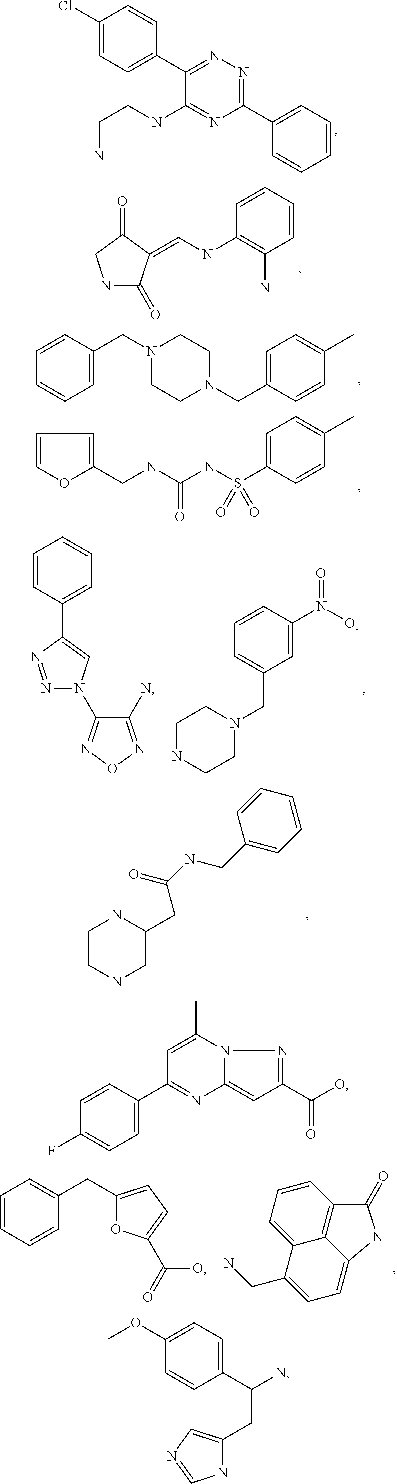

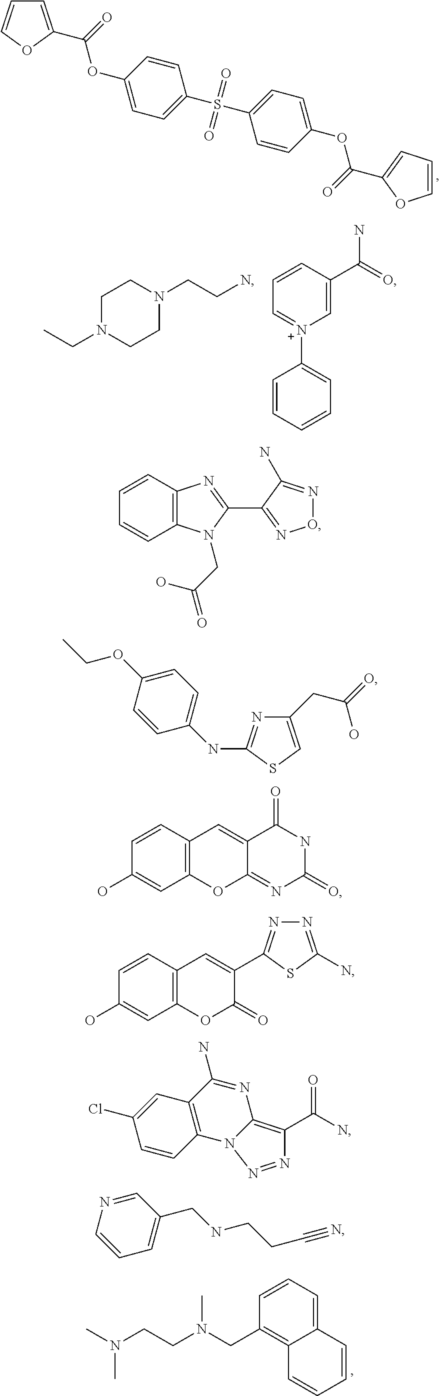

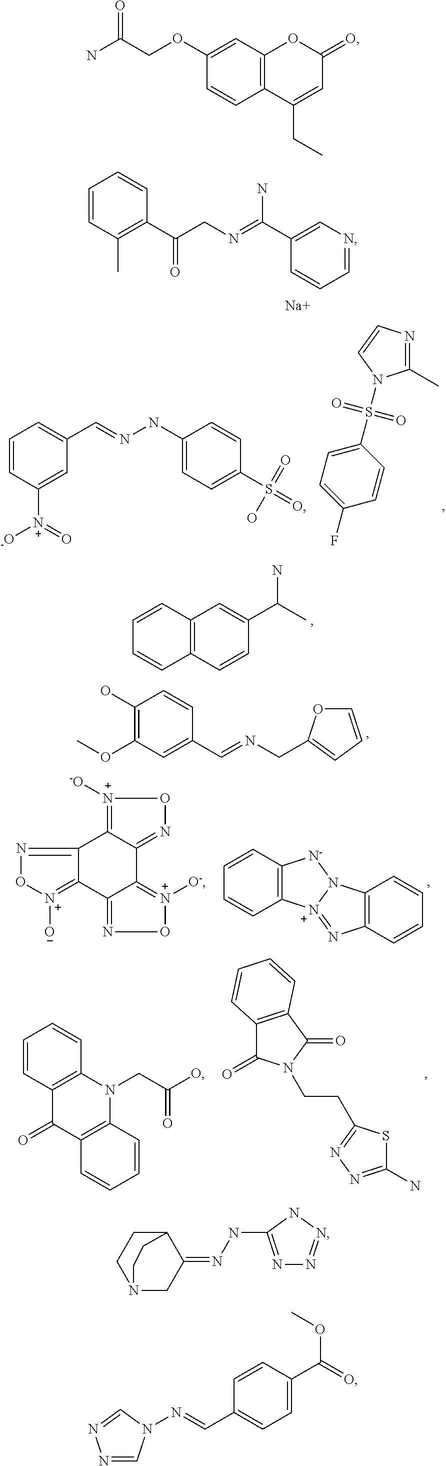

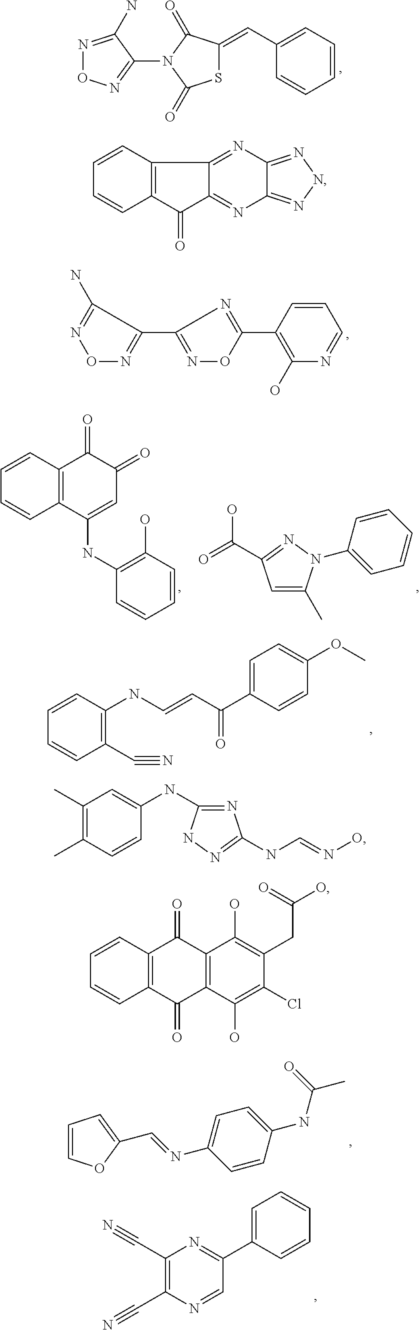

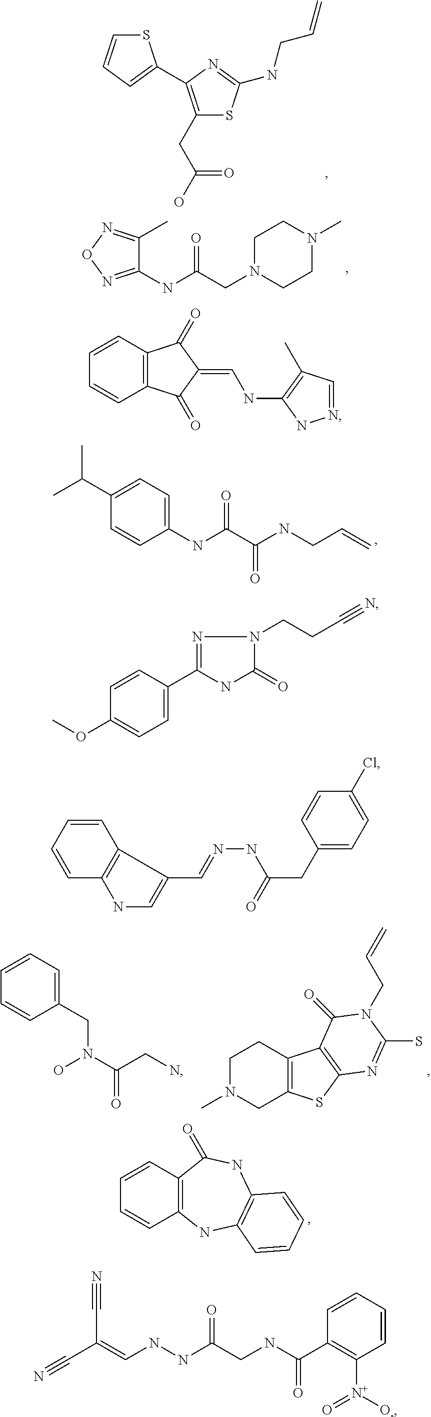

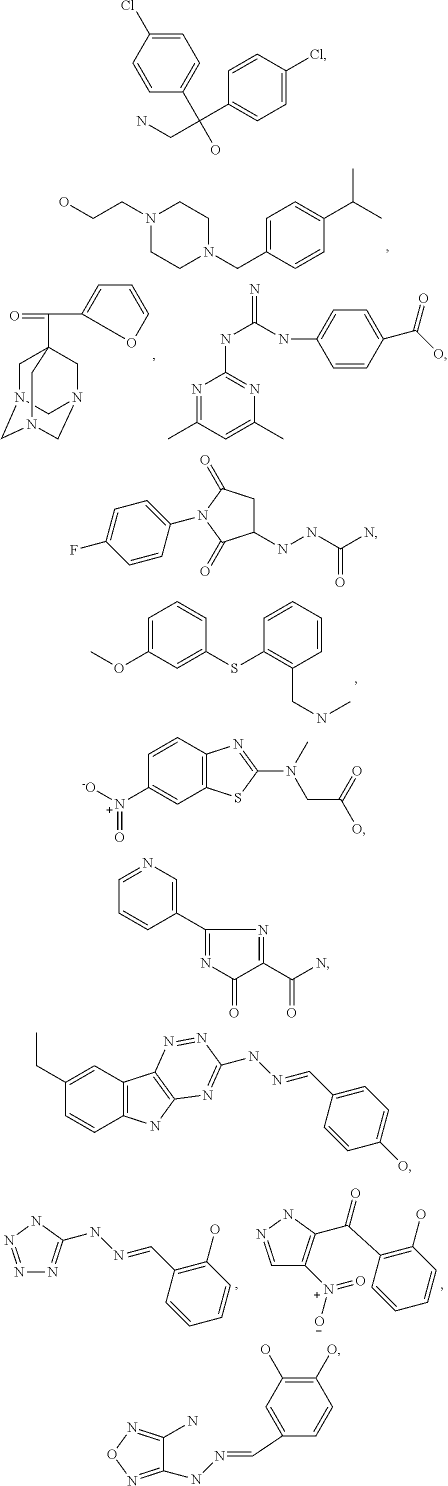

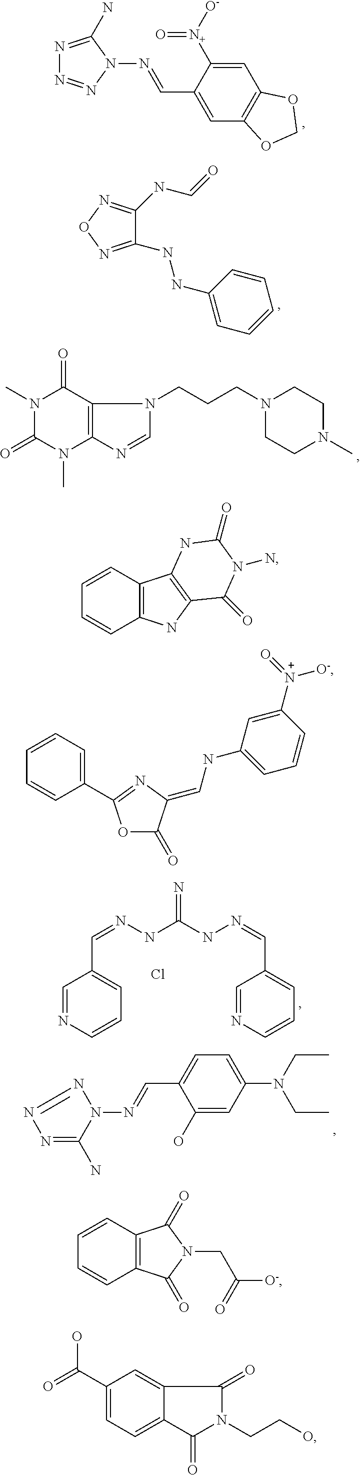

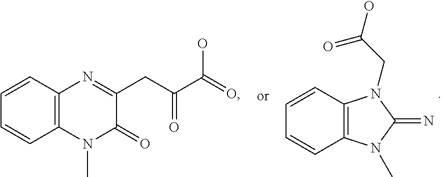 [SMILES: Cn1c(=O)c(CC(=O)C(=O)O)nc2ccccc21 ; "or" ; Cn1c(=N)n(CC(=O)O)c2ccccc21]

In another embodiment, the second inhibitor is any combination of the above-described second inhibitor compounds.

In any of the embodiments above, the term “alkyl” implies a straight-chain or branched alkyl containing, for example, from 1 to 6 carbon atoms, e.g., from 1 to 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., fluoro C₁-C₆ alkyl. The alkyl may be substituted or unsubstituted, as described herein.

In any of the embodiments above, the term “aryl” refers to a mono, bi, or tricyclic carbocyclic ring system that may have one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hückel's Rule, wherein n=1, 2, or 3. The aryl may be substituted or unsubstituted, as described herein.

In any of the embodiments above, the term “heteroaryl” refers to an aryl as defined above in which at least one, preferably 1 or 2, of the carbon atoms of the aromatic carbocyclic ring is replaced by N, O or S atoms. Examples of heteroaryl include pyridyl, furanyl, pyrrolyl, quinolinyl, thiophenyl, indolyl, imidazolyl and the like.

In other aspects, any substituent that is not hydrogen (e.g., C₁-C₆ alkyl, C₂-C₆ alkenyl, C₃-C₆ cycloalkyl, or aryl) may be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxyl, cyano, nitro, and others, the aromatic ring hydrogen is replaced with the substituent and this may take place in any of the available hydrogens, e.g., 2, 3, 4, 5, and/or 6-position wherein the 1-position is the point of attachment of the aryl group in the compounds, salts, solvates, or stereoisomers of the present invention. Suitable substituents include, e.g., halo, alkyl, alkenyl, alkynyl, hydroxyl, nitro, cyano, amino, alkylamino, alkoxyl, aryloxyl, aralkoxyl, carboxyl, carboxyalkyl, carboxyalkyloxy, amido, alkylamido, haloalkylamido, aryl, heteroaryl, and heterocycloalkyl. In some instances, the substituent is at least one alkyl, halo, and/or haloalkyl (e.g., 1 or 2).

The first and/or second inhibitor can be formulated into a composition, such as a pharmaceutical composition, and can be either together in the same composition or in separate compositions. In this regard, an embodiment of the invention provides pharmaceutical compositions comprising the first and/or second inhibitor and a pharmaceutically acceptable carrier.

The pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular first and/or second inhibitor, as well as by the particular method(s) used for administration. Accordingly, there are a variety of suitable formulations of the pharmaceutical compositions of the invention. Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The first and/or second inhibitor may be provided in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The concentration of the first and/or second inhibitor in the pharmaceutical formulations may vary, e.g., from less than about 1%, usually at or at least about 10%, to as much as about 20% to about 50% or more by weight, and may be selected primarily by fluid volumes, and viscosities, in accordance with the particular mode of administration selected.

Methods for preparing administrable (e.g., parenterally administrable) compositions are known or apparent to those skilled in the art and are described in more detail in, for example, Remington: The Science and Practice of Pharmacy, Lippincott Williams & Wilkins; 21st ed. (May 1, 2005).

The following formulations for oral, aerosol, parenteral (e.g., subcutaneous, intravenous, intraarterial, intramuscular, intradermal, interperitoneal, and intrathecal), and topical administration are merely exemplary and are in no way limiting. More than one route may be used to administer the first and/or second inhibitor, and in certain instances, a particular route may provide a more immediate and more effective response than another route.

Formulations suitable for oral administration may comprise or consist of (a) liquid solutions, such as an effective amount of the first and/or second inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms may be of the ordinary hard or softshelled gelatin type containing, for example, surfactants, lubricants, and inert fillers, such as lactose, sucrose, calcium phosphate, and corn starch. Tablet forms may include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms may comprise the first and/or second inhibitor in a flavor, usually sucrose and acacia or tragacanth, as well as pastilles comprising the first and/or second inhibitor in an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which may contain antioxidants, buffers, bacteriostats, and solutes that render the formulation isotonic with the blood of the intended recipient, and aqueous and nonaqueous sterile suspensions that may include suspending agents, solubilizers, thickening agents, stabilizers, and preservatives. The first and/or second inhibitor can be administered in a physiologically acceptable diluent in a phannaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which may be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain, for example, from about 0.5% to about 25% by weight of the first and/or second inhibitor in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and may be stored in a freeze-dried (lyophilized) condition requiring only the addition of the sterile liquid excipient, for example, water, for injections, immediately prior to use. Extemporaneous injection solutions and suspensions may be prepared from sterile powders, granules, and tablets of the kind previously described.

Injectable formulations are in accordance with an embodiment of the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986), incorporated by reference herein).

Topical formulations, including those that are useful for transdermal drug release, are well known to those of skill in the art and are suitable in the context of embodiments of the invention for application to skin. The first and/or second inhibitor, alone or in combination with other suitable components, may be made into aerosol formulations to be administered via inhalation. These aerosol formulations may be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

An “effective amount” or “an amount effective to treat” refers to a dose that is adequate to prevent or treat Mtb infection in a mammal. Amounts effective for a therapeutic or prophylactic use will depend on, for example, the stage and severity of the Mtb being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that the Mtb infection could require prolonged treatment involving multiple administrations, perhaps using the first and/or second inhibitor in each or various rounds of administration. By way of example and not intending to limit the invention, the dose of the first and/or second inhibitor may be about 0.001 to about 1000 mg/kg body weight of the mammal being treated/day, from about 0.01 to about 10 mg/kg body weight/day, about 0.01 mg to about 1 mg/kg body weight/day.

For purposes of the invention, the amount or dose of the first and/or second inhibitor administered should be sufficient to effect a therapeutic or prophylactic response in the mammal over a reasonable time frame. For example, the dose of the first and/or second inhibitor should be sufficient to treat or prevent disease in a period of from about 2 hours or longer, e.g., about 12 to about 24 or more hours, from the time of administration. In certain embodiments, the time period could be even longer. The dose will be determined by the efficacy of the particular first and/or second inhibitor and the condition of the mammal (e.g., human), as well as the body weight of the mammal (e.g., human) to be treated.

In addition to the aforedescribed pharmaceutical compositions, the first and/or second inhibitor may be formulated as inclusion complexes, such as cyclodextrin inclusion complexes, or liposomes. Liposomes may serve to target the first and/or second inhibitor to a particular tissue. Liposomes also may be used to increase the half-life of the first and/or second inhibitor. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

The delivery systems useful in the context of embodiments of the invention may include time-released, delayed release, and sustained release delivery systems such that the delivery of the pharmaceutical composition occurs prior to, and with sufficient time to cause, sensitization of the site to be treated. The pharmaceutical composition can be used in conjunction with other therapeutic agents or therapies. Such systems can avoid repeated administrations of the pharmaceutical composition, thereby increasing convenience to the mammal and the physician, and may be particularly suitable for certain composition embodiments of the invention.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems may be used, some of which are adapted for implantation.

One of ordinary skill in the art will readily appreciate that the first and/or second inhibitor of the invention may be modified in any number of ways, such that the therapeutic or prophylactic efficacy of the first and/or second inhibitor is increased through the modification. For instance, the first and/or second inhibitor may be conjugated either directly or indirectly through a linking moiety to a targeting moiety. The practice of conjugating compounds, e.g., first and/or second inhibitor, to targeting moieties is known in the art. See, for instance, Wadwa et al., J. Drug Targeting 3: 111 (1995) and U.S. Pat. No. 5,087,616.

The first and/or second inhibitor may be modified into a depot form, such that the manner in which the first and/or second inhibitor is released into the body to which it is administered is controlled with respect to time and location within the body (see, for example, U.S. Pat. No. 4,450,150). Depot forms of first and/or second inhibitor may be, for example, an implantable composition comprising the first and/or second inhibitor and a porous or non-porous material, such as a polymer, wherein the first and/or second inhibitor are encapsulated by or diffused throughout the material and/or degradation of the non-porous material. The depot is then implanted into the desired location within the body and the first and/or second inhibitor are released from the implant at a predetermined rate.

The first and second inhibitor can be coadministered to the mammal. By “coadministering” is meant administering the first and/or second inhibitor sufficiently close in time such that the first and/or second inhibitor can enhance the effect of one another. In this regard, the first inhibitor can be administered first and the second inhibitor can be administered second, or vice versa. Thus, in an embodiment, the first and second inhibitors are administered sequentially. Alternatively, the first and second inhibitor can be administered simultaneously. In an embodiment, the first and second inhibitors are administered simultaneously.

The mammal referred to herein may be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount or any level of treatment or prevention of Mtb in a mammal. Furthermore, the treatment or prevention provided by the inventive method can include treatment or prevention of one or more conditions or symptoms of the disease, i.e., Mtb, being treated or prevented. Also, for purposes herein, “prevention” can encompass delaying the onset of the disease, or a symptom or condition thereof.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following example further illustrates the invention but, of course, should not be construed as in any way limiting its scope.

### Example

This example demonstrates use of an inhibitor of heme degradation enhances host resistance to M. tuberculosis, in accordance with embodiments of the invention.

While HO-1 expression has been shown to be induced in the lungs of mice after infection with M. tuberculosis, studies addressing the precise role of HO-1 in mycobacterial infection in the murine model have yielded contradictory results. Some labs have reported that congenitally HO-1-deficient mice are more susceptible to experimental M. tuberculosis as well as M. avium infections. However, in the absence of infection, these mutant mice display high in utero mortality (around 80%), and the surviving animals display prominent abnormalities in hematopoietic cells, especially in macrophages, the major cell population infected by M. tuberculosis. Macrophages in uninfected congenitally HO-1-deficient mice are aberrantly short-lived, and this is reflected in histopathological and functional alterations in lymphoid organs. Thus, because of their baseline genetic abnormalities, HO-1-deficient mice may not be the best tool for assessing the role of HO-1 during M. tuberculosis infection.

C57BL/6 and T-cell receptor α-deficient (TCR-α^{-/-}) mice (purchased from Taconic Farms, Germantown, N.Y., USA). All animals were housed at biosafety level 2 (BSL-2) and BSL-3 animal facilities at the National Institute of Allergy and Infectious Diseases (NIAID), National Institutes of Health (NIH), and all experiments utilized protocols approved by the NIAD Animal Care and Use Committee. Mice were aerosol-infected with 100 CFU of M. tuberculosis strain H37Rv using an aerosol chamber (Glas Col, Terre Haute, Ind., USA). Determination of bacterial loads was performed by culturing serial dilutions of tissue homogenates in 7H11 medium (Sigma-Aldrich, St. Louis, Mo., USA) supplemented with oleic acid-albumin-dextrose-catalase (BD Biosciences, San Diego, Calif., USA). The heme-oxygenase inhibitor SnPPIX (Frontier Scientific, Logan, Utah, USA) was administered by daily peritoneal injection (5 mg/kg/mouse). SnPPIX was dissolved in 0.1 M NaOH aqueous solution, and then diluted in a 10× phosphate-buffered saline (PBS), and the pH of the solution adjusted to 7.0 to 7.4. Aliquots of SnPPIX were frozen at −80° C. and thawed immediately prior to inoculation.

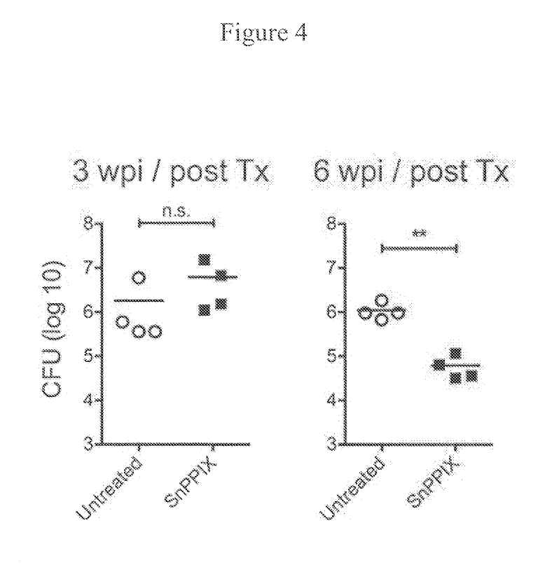

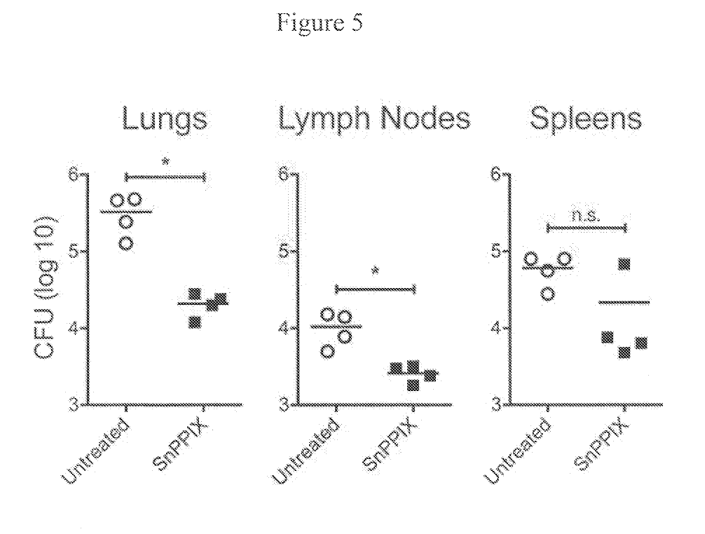

SnPPIX was given to C57BL/6 animals by daily intraperitoneal injection beginning on the same day as the aerosol M. tuberculosis infection. As seen in FIG. 4, SnPPIX induced a highly significant reduction in pulmonary bacterial load that was evident at 6 weeks post-infection (wpi), but not at 3 wpi. As seen in FIG. 5, a similar reduction was achieved when SnPPIX treatment was initiated at 4 wpi, and bacterial load was measured 3 weeks later (3 wks post Tx). The effects of SnPPIX administration were more prominent in the lungs than in mediastinal lymph nodes or spleens.

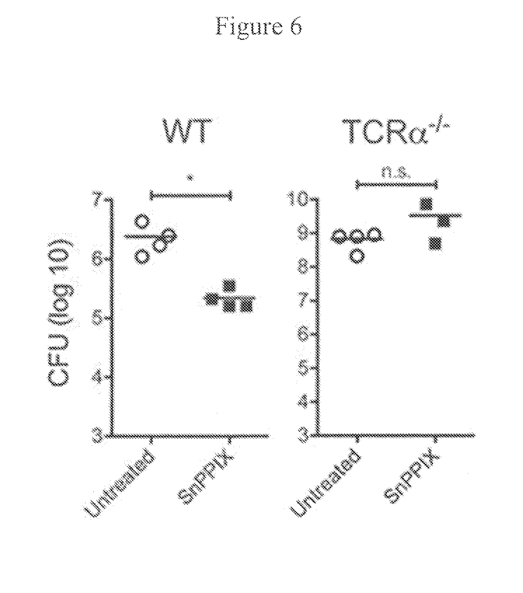

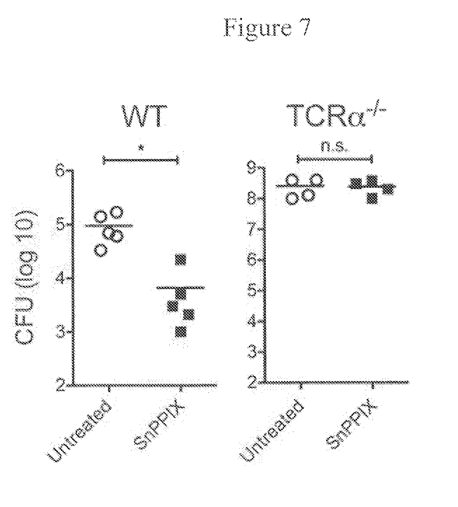

T-cell receptor α-deficient (TCR-α^{-/-}) mice lack conventional TCR-αβ+CD4 and CD8 T cells. To determine if T lymphocytes are required for the SnPPIX activity, SnPPIX was administered to M. tuberculosis-infected C57BL/6 mice (WT) and TCR-α^{-/-} mice beginning at 4 weeks post-infection. SnPPIX treatment failed to protect the infected TCR-α^{-/-} mice, which presented similar mortality kinetics as TCR-α^{-/-} mice not treated with SnPPIX. The results of parallel experiments, in which mice were euthanized two weeks after SnPPIX treatment initiation are shown in FIG. 6. This figure shows that SnPPIX-treated infected TCR-α^{-/-} mice displayed bacterial loads indistinguishable from those of untreated infected TCR-α^{-/-} mice, and WT mice treated with SnPPIX showed a highly significant reduction in mycobacterial load compared to WT mice not treated with SnPPIX. To control for the higher bacterial burden expected in TCR-α^{-/-} mice, a separate set of experiments were conducted in which SnPPIX administration was initiated on the same day as M. tuberculosis infection, and the pulmonary bacterial load was evaluated 6 weeks post-infection. As seen in FIG. 7, WT mice receiving SnPPIX displayed a significant reduction in pulmonary bacterial loads, while no difference in bacterial loads was observed among the surviving TCR-α^{-/-} mice. These results suggest that the efficacy of SnPPIX on M. tuberculosis infection is dependent on host T cells, and not necessarily a direct effect of the inhibitor on the bacteria.

Experiments were performed to determine if SnPPIX is toxic for M. tuberculosis by targeting the bacterium's own heme degrading enzyme, MhuD. Two different assays were employed to determine whether SnPPIX is toxic for Mtb in culture. In the first assay, the minimum concentration of drug inhibiting 99% (MIC99) of bacterial growth was determined using a previously described micro-dilution broth method (Wong et al., Antimicrob. Agents Chemother. 55: 2515-2522 (2011), incorporated by reference herein), employing glycerol-alanine-salts (GAST) medium or GAST medium supplemented with either iron (GASTFe) or 10 μM hemin (GAST+hemin). Briefly, M. tuberculosis strain H37Rv was adapted to the Fe-sufficient and Fe-deficient media for 3 weeks with repeated sub-culturing once every week in fresh medium. The bacteria were then allowed to grow to an optical density (OD650) of 0.2 to 0.3, diluted to a final OD650 of 0.0002 (1:1000 of parent culture) and distributed into a 96-well plate. SnPPIX was then added at 125 μM, 62.5 μM, 31.25 μM, 15.63 μM, 7.81 μM, 3.9 μM, 3.95 μM, 0.98 μM, 0.49 μM, 0.24 μM and 0.12 μM to duplicate plates with each compound dilution further set-up in duplicate rows per plate. Cultures were incubated for 28 days at 37° C. and the lowest concentration of drug that inhibited visible growth was determined. Isoniazid was used as a positive control for anti-bacterial activity. Growth inhibition was also assessed in a second nitrosative stress assay wherein green fluorescent protein (GFP)-expressing M. tuberculosis/pMSP12 bacteria were adapted to 7H9 medium containing 250 μM butyrate (as the carbon source) at pH 6.0 for 14 days. The inhibition assay was set up as described above but in the presence or absence of 100 μM NaNO₂ rather than iron or hemin. On day 21 of culture, fluorescence was measured in an ENVISION multiplate reader (Perkin Elmer, Waltham, Mass., USA) and the MIC95 values calculated as the drug concentration giving 95% inhibition of the fluorescent signal (fluorescence read in the presence of rifampicin was used as positive control −100% inhibition, while fluorescence in the presence of DMSO vehicle was used as negative control −0% inhibition). Pyrazinamide is only active at low pH environment and therefore, inhibition of bacterial growth in its presence was used as a positive control for confirming maintenance of the low pH of the medium through the duration of the assay.

Bacteria were cultured in either iron-containing or iron-free GAST (glycerolalanine-salts-Tween 80) liquid medium in the presence of increasing concentrations of SnPIX over a 28-day period. No inhibition of bacterial growth was observed, even when the bacteria were cultured in the presence of 125 μM SnPPIX in complete medium, while toxicity was observed when the bacteria were cultured in iron-free medium with 125 μM SnPPIX. Addition of 10 μM hemin reversed this effect, suggesting that attenuation was unrelated to the inhibition of MhuD activity by SnPPIX.

When exposed to adverse conditions, such as low pH and oxygen concentrations, as well as to reactive oxygen or nitrogen species, M. tuberculosis undergoes changes in gene expression and metabolism that promote its survival in the harsh phagosomal environment of activated macrophages (Russell D G, Infection, Immunol. Rev., 240: 252-268 (2022), incorporated by reference herein). In order to test whether such conditions might promote bacterial sensitivity to SnPPIX, M. tuberculosis was cultured in low-pH 7H9 medium in the presence of 100 μM sodium nitrite to simulate both acid and nitrosative stress from the intramacrophage compartment. Even at SnPPIX concentrations as high as 125 μM, no inhibition of bacterial growth was observed over a 21-day period in either the presence or absence of nitrite. See Table 1, below.

TABLE 1
Effect of SnPPIX on M. tuberculosis Growth
Experiment 1 - day 28 MIC₉₉

| | SnPPIX (μM) | Isoniazid (μM) |
|---|---|---|
| GASTFe | >125 | 0.2 |
| GAST | 125 | 0.1 |
| GAST + 10 μM Hemin | >125 | 0.1 |

Experiment 2 - day 21 MIC₉₅

| | SnPPIX (μM) | Rifampicin (μM) |
|---|---|---|
| 7H9-butyrate | >125 | 0.04 |
| 7H9-butyrate − 100 μM NaNO₂ | >125 | 0.04 |

Construction of the expression vector, pET22b-MhuD with a C-terminal His₆tag, and the expression and purification of M. tuberculosis MhuD have been previously described (Chim N. et al., J. Mol. Biol., 395: 595-608 (2010), incorporated by reference herein). In brief, MhuD was overexpressed in BL21-Gold (DE3) Escherichia coli. Cells were resuspended in 50 mM Tris/HCl pH 7.4, 350 mM NaCl and 10 mM imidazole and lysed by sonication. The cell supernatant was loaded onto a Ni₂₊-charged 5 mL HITRAP chelating column and washed with resuspension buffer. Fractions of eluted MhuD (between 50 and 100 mM imidazole) were collected and concentrated. Apo-MhuD was further separated on a S75 gel filtration column (GE Healthcare, Little Chalfont, UK) with 20 mM Tris, pH 8, and 10 mM NaCl before a final purification step with an ion exchange column (HITRAP Q HP, 5 mL) where homogeneous apo-MhuD eluted at 150 mM NaCl. Recombinant human heme oxygenase-1 variant G139A (hHO-1 G139A) clone was a gift from Dr. Thomas L. Poulos from the University of California, Irvine and was purified as previously described (Wilks A. et al., J. Biol. Chem., 268: 22357-22362 (1993); Liu Y et al., J. Biol. Chem., 275: 34501-34507b(2000), incorporated by reference herein).

To determine if SnPPIX blocks MhuD, the M. tuberculosis heme degrading enzyme, SnPPIX was dissolved in 300 μl of 0.1 M NaOH before dilution into 50 mM (hydroxymethyl)aminomethane (TRIS) pH 7.4, 150 mM NaCl. The pH was readjusted back to 7.4 with 1 M HCl. Hemin was prepared by dissolving hemin chloride (Sigma-Aldrich, St. Louis, Mo., USA) in 0.1 M NaOH before the addition of 1 M TRIS pH 7.4 and dilution into 50 mM TRIS pH 7.4, 150 mM NaCl. To produce the MhuD-SnPPIX complex, SnPPIX was gradually added to 0.1 M Apo-MhuD in a 1:1 molar ratio. The mixture was incubated overnight at 4° C. and exchanged into 50 mM Tris pH 7.4, 150 mM NaCl via a 5 mL HITRAP desalting column (GE Healthcare Life Sciences, Pittsburgh, Pa., USA). MhuD-heme was prepared as previously reported (Tullius M V et al, Proc. Natl. Acad. Sci. USA, 108:5051-5056 (2011), incorporated by reference herein). Briefly, heme was gradually added to 0.1 M MhuD in a 1.2:1 molar ratio before overnight incubation at 4° C. In all cases, excess heme was removed using a 5 mL HITRAP desalting column and the eluted protein concentration was determined by LOWRY assay. The human HO-1 variant G139A (hHO-1 G139A) was used as a positive control for heme degradation by the host enzyme as its reaction rate is attenuated by 58% (Liu Y, et al., J. Biol. Chem., 275: 34501-34507 (2000), incorporated by reference herein), allowing for the observation of single turnover heme degradation within a similar time period as MhuD. The heme degradation reaction for hHO-G139A-heme was carried out in a similar manner as that for MhuD-heme. In all assays, sodium ascorbate was added as an electron donor to initiate the heme degradation process, as previously described (Chim N. et al., J. Mol. Biol., 395: 595-608 (2010), incorporated by reference herein). The reaction was monitored by UV/vis spectroscopy by collecting spectra between 300-700 nm at various time intervals and observing the decrease of the SORET peak over time to determine heme degradation. For the SnPPIX/heme competition assays, 2 μM SnPPIX was incubated with either 5 μM MhuD-heme or 5 μM apo-MhuD, as well as 5 μM hHOG139A-heme or 5 μM apo-hHO-1 G139A for 1 hour before addition of 10 mM sodium ascorbate to initiate the reaction. To remove the absorbance interference of SnPPIX the difference spectra (ΔAbsorbance) were calculated by subtracting the reaction spectra obtained without enzyme from those spectra obtained for the 5 μM enzyme-heme reaction.

Single cell suspensions were obtained from Mtb-infected lungs as previously described (Mayer-Barber K D et al., Nature, 511: 99-103 (2014), incorporated by reference herein). Cells were stimulated with PMA (10 ng/ml) and ionomicin (1 μg/ml) in the presence of brefeldin A and monensin for 5 hours prior to staining with specific fluorochrome-labelled antibodies and a fixable fluorescent viability dye (Molecular Probes/Thermo Fisher Scientific). The antibodies employed (obtained from either Affimetrix/ebioscience or BD biosciences (San Diego, Calif., USA)) were directed against CD3 (clone 145-2C11), CD4 (clone GK1.5), CD8 (clone 53-6.7) and IFN-γ (clone XMG 1.2). All samples were acquired on LSRII flow cytometers (BD Biosciences) and analyzed utilizing FLOWJO software (FlowJo LLC, Ashland, Oreg., USA).

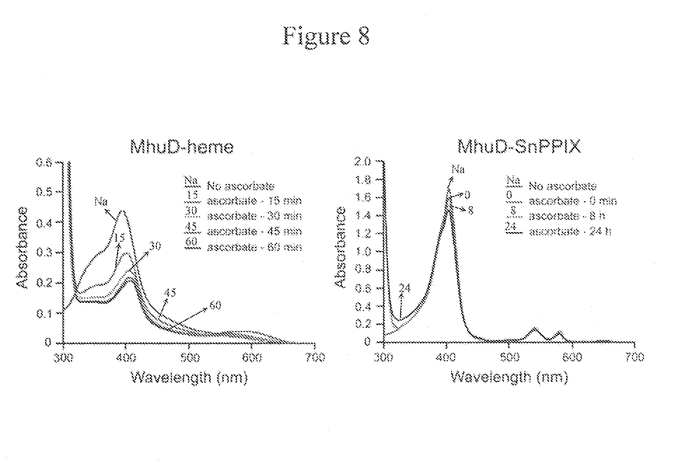

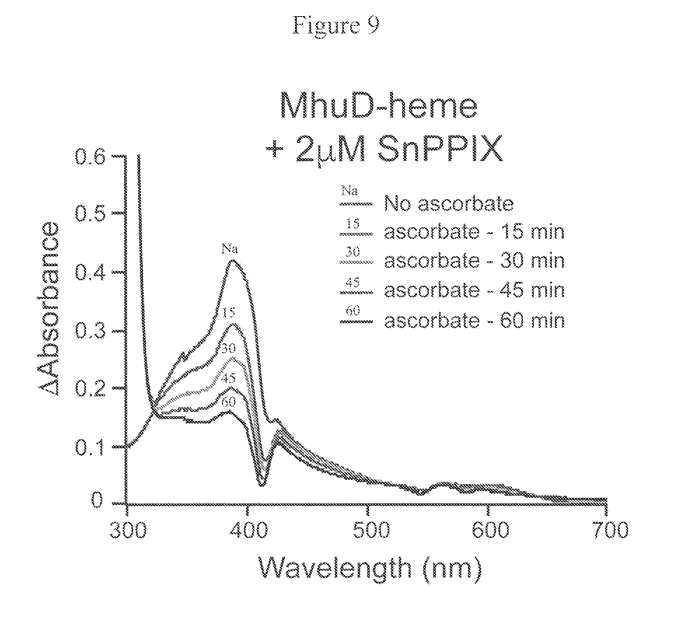

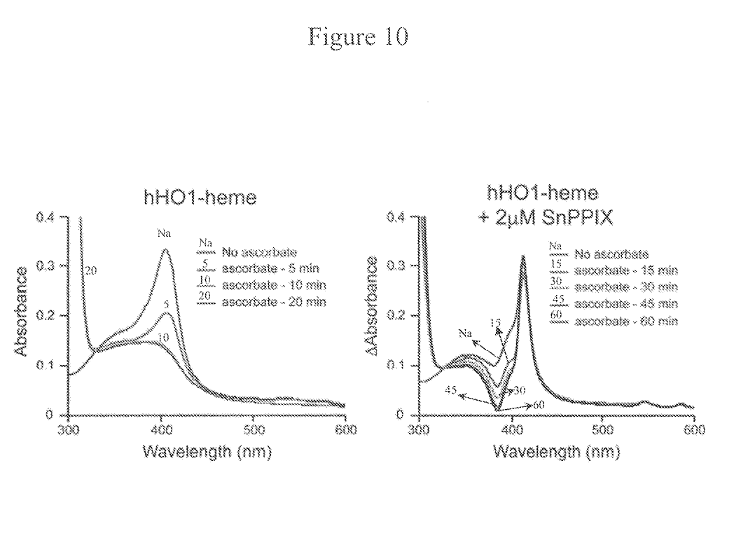

The following experiments were conducted to determine whether SnPPIX could be directly degraded by M. tuberculosis MhuD or inhibit its heme-cleaving activity. Degradation of heme and SnPPIX by MhuD was monitored by UV-visible spectroscopy every 5 minutes for 1 hour for heme and for a period of 24 hours for SnPPIX. The results shown in FIG. 8, where each line represents a different time point, indicate that heme degrades quickly while there appears to be no SnPPIX degradation even after 24 hours. These results suggest that SnPPIX is not degraded by MhuD. SnPPIX does not appear to have an effect on the heme degradation by MhuD, as seen in FIG. 9, where degradation of heme in the presence of 2 μM SnPPIX was monitored every 5 minutes for 1 hour. Data shown in FIG. 9 are expressed as Δabsorbance ([absorbance of 5 μM MhuD-heme+2 μM SnPPIX]−[absorbance of 2 μM SnPPIX]) for each time point in order to correct for the absorbance in the presence of SnPPIX. In contrast, as seen in FIG. 10, the heme-degrading activity of mammalian HO-1 was blocked by 2 μM SnPPIX. Heme degradation by recombinant human HO-1-G139A (hHO-1) in the absence or presence of 2 μM SnPPIX was monitored every 5 minutes for 1 hour. The left panel of FIG. 10 shows the measured absorbance, and the right panel of FIG. 10 shows the change in absorbance (Δabsorbance, as described above). All experiments for FIG. 8, FIG. 9, and FIG. 10 were performed in triplicate. These results suggest that the in vivo effects of SnPPIX on M. tuberculosis infection are unlikely to be due to a direct effect of the compound on the bacterium itself.

mRNA was extracted from lungs of M. tuberculosis-infected and naive mice by using Trizol reagent (Invitrogen/Thermo Fisher Scientific, Waltham, Mass., USA), and RNEASY minikits (Qiagen, Hilden, Germany). cDNA was reverse transcribed using 1 μg of RNA, SUPERSCRIPT II reverse transcriptase, and random primers (all from Invitrogen/Thermo Fisher Scientific). SYBR green and 7900HT fast real-time PCR systems (Applied Biosystems/Thermo Fisher Scientific) were employed for real-time PCRs. The relative expression of HO-1 in M. tuberculosis-infected mouse lungs was calculated using the ΔΔᵣ, (cycle threshold) method, normalizing mRNA expression in each sample to that of β-actin, and further comparing them in relation to expression in uninfected naïve mouse lungs. The primer sequences used are provided in Table 2, below.

TABLE 2
Primer Nucleotide Sequences

| Name | Orientation | Nucleotide Sequence | SEQ ID NO: |
|---|---|---|---|
| Murine | Forward primer | AGC TGC GTT TTA CAC CCT TT | 1 |
| Actb | Reverse primer | AAG CCA TGC CAA TGT TGT CT | 2 |
| Murine | Forward primer | GCC ACC AAG GAG GTA CAC AT | 3 |
| Hmox1 | Reverse primer | GCT TGT TGC GCT CTA TCT CC | 4 |
| Mtb Rv3592 | Reverse primer | TTA TGC AGT CTT GCC GGT CC | 5 |
| (MhuD)- | | | |
| cDNA | | | |
| Mtb Rv3592 | Forward primer | AAC GCT ACT TCG TGG TGA CA | 6 |
| (MhuD)- | Reverse primer | CGT CAA GCA CGA CCT CGA AT | 7 |
| real time | | | |
| PCR | | | |

For Western blotting, M. tuberculosis-infected and naïve mouse lungs were perfused with PBS and homogenized in PBS containing Complete Ultra protease inhibitor cocktail (Roche, Basel, Switzerland) and 2 mM phenylmethylsulfonyl fluoride (Sigma Aldrich). The protein concentrations from all samples were normalized, and then reducing buffer (Pierce/Thermo Fisher Scientific) was added to samples prior to incubation for 5 min at 95° C. for protein denaturation. Samples were separated in Mini-Protean TGX gels (Bio-Rad, Hercules, Calif., USA) and transferred to polyvinylidene difluoride membranes prior to staining with anti-mouse HO-1 (SPA-895; Enzo Life Sciences, Farmingdale, N.Y., USA) or anti-mouse glyceraldehyde-3-phosphate dehydrogenase (GAPDH; ab9485; Abcam, Cambridge, Mass., USA) and anti-rabbit IgG conjugated to horseradish peroxidase (catalog number 7074; Cell Signaling Technology, Danvers, Mass., USA).

Differences between groups were statistically evaluated by using an unpaired Student t test (based on a parametric distribution of the data) within Prism software (GraphPad, San Diego, Calif., USA), and differences were considered significant when P was ≤0.05.

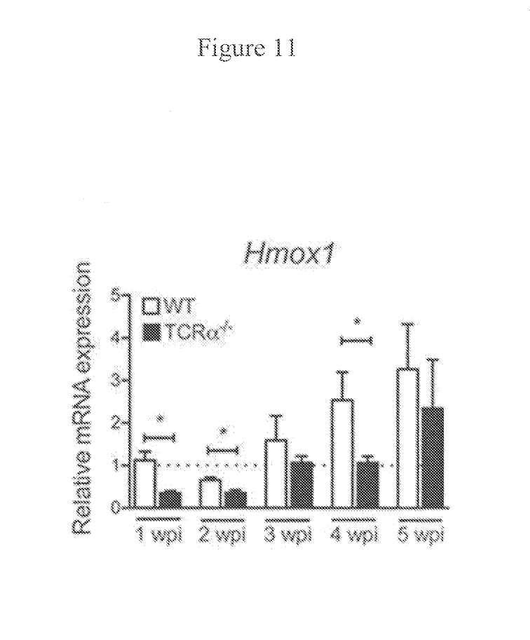

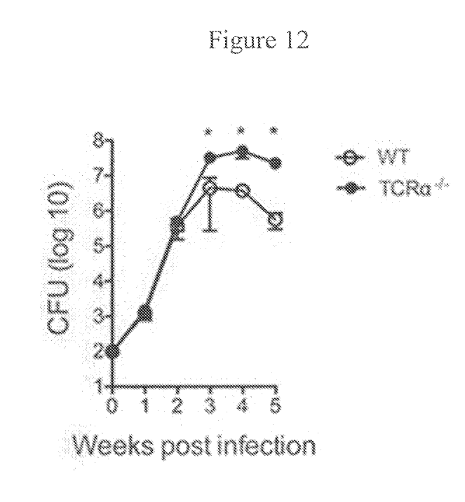

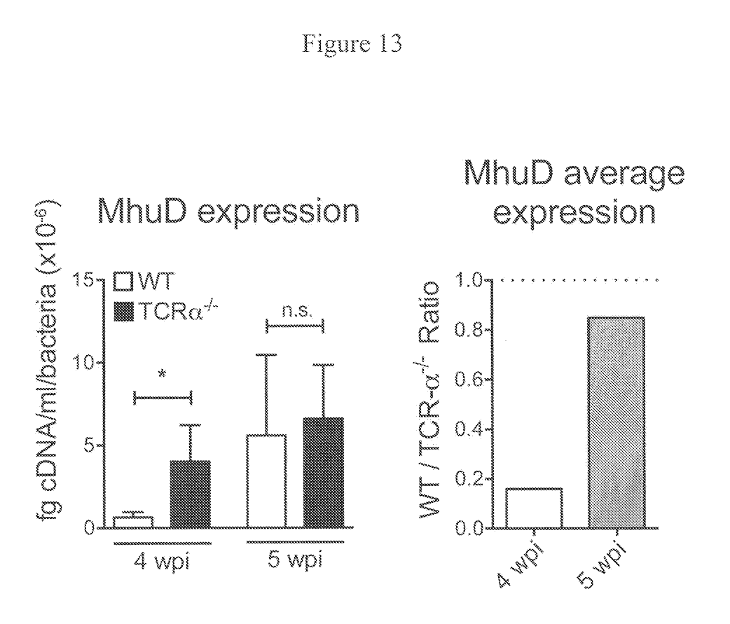

The following experiment was conducted to determine if the induction of the host HO-1 is altered in T cell deficient mice. In lungs of WT mice, increases in HO-1 were not detected until 3 wpi, and the protein, as measured by Western blotting, was first evident at the same time point. In lungs of TCR-α^{-/-} mice, HO-1 gene and protein expression were delayed until 4 or 5 wpi and were reduced relative to that observed in lungs of WT mice. FIG. 11 presents a graph of the relative mRNA expression of HO-1 measured by real time polymerase chain reaction (PCR) in lungs of M. tuberculosis-infected WT and TCR-α^{-/-} mice at 1, 2, 3, 4, and 5 wpi. As shown in FIG. 12, the reduced levels of HO-1 mRNA and protein expression in TCR-α^{-/-} mice occurred despite the increased bacterial loads present in the TCR-α^{-/-} mice. In contrast, expression of bacterial MhuD mRNA was increased in the lungs of infected TCR-α^{-/-} mice when compared with WT mice (see FIG. 13), reinforcing the finding that M. tuberculosis MhuD is unaffected by SnPPIX and plays no role in the phenomena observed.

Together, the above results suggest that M. tuberculosis infection is refractory to SnPPIX treatment in T cell-deficient mice because of reduced pulmonary expression of host HO-1. The latter could result from either impaired recruitment of enzyme-expressing cells to the lungs, or defective induction of enzyme synthesis because of the absence of a T cell response. In this regard, macrophages and monocytes rather than T cells have been shown to be the major source of HO-1 in infected lungs of WT mice as well as human lungs (Scharn C R et al., J. Immunol., 196: 4641-4649 (2016), incorporated by reference herein). While purified bone marrow-derived macrophage cultures can produce HO-1 in response to M. tuberculosis infection in the absence of T cells (Andrade B B et al., J. Immunol. 195: 2763-2773 (2015), incorporated by reference herein), it is possible that the infected tissue macrophage subpopulations in the lungs of M. tuberculosis-exposed mice require additional T cell activation signals to achieve optimal enzyme expression in vivo.

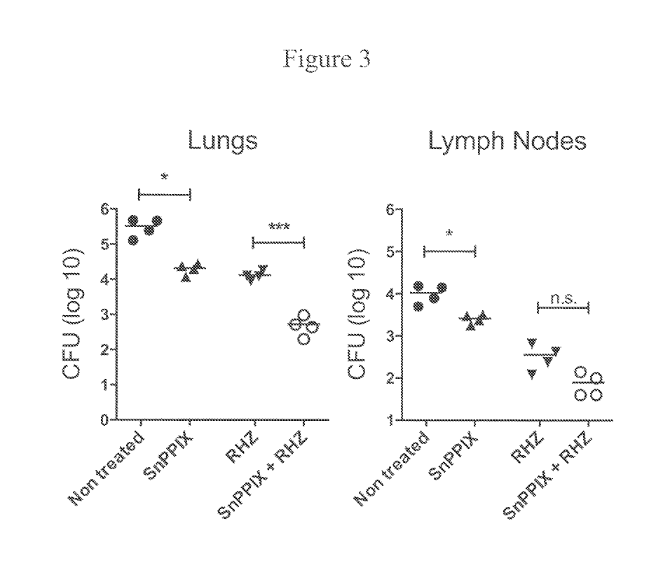

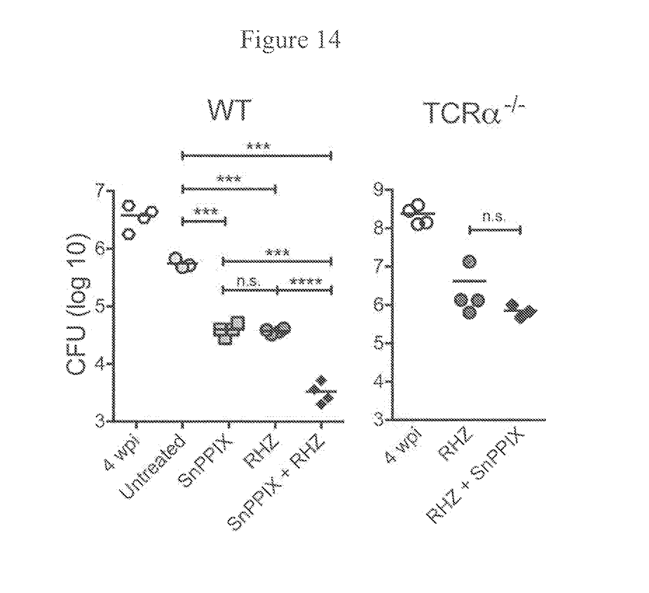

Experiments were then performed to evaluate whether adjunctive administration of SnPPIX could enhance the efficacy of conventional tuberculosis treatment. Starting at 28 days post-infection, mice were: left untreated; intraperitoneally (i.p.) treated with SnPPIX (5 mg/kg) daily; orally treated with pyrazinamide, rifampicin, and isoniazid (“RHZ”) five times per week, or orally treated with RHZ five times per week and i.p. treated with SnPPIX daily. Mice were euthanized 21 days after initiation of treatment, and bacterial loads in lungs and draining mediastinal lymph nodes were assessed. FIG. 3 presents results on the lungs and lymph nodes of WT mice, and FIG. 14 presents the results on the lungs of WT mice and the lungs of TCR-α^{-/-} mice. The RHZ and SnPPIX treatments each resulted in an approximate 1-log reduction in pulmonary bacterial loads below those in untreated infected mice.

The data suggests that daily intraperitoneal administration of SnPPIX to WT mice with established M. tuberculosis infection results in diminished bacterial loads in lungs and mediastinal lymph nodes in comparison to non-treated mice, comparable to that achieved with conventional antibiotic treatment (isoniazid, pyrazinamide and rifampicin). The combined administration of SnPPIX with antibiotics to WT mice resulted in enhanced reductions in pulmonary bacterial loads compared to those seen following standard chemotherapy or SnPPIX treatment alone and without obvious side effects to the animals. As seen in the right panel of FIG. 14, the combined administration of SnPPIX with antibiotics to TCR-α^{-/-} mice failed to enhance RHZ efficacy.

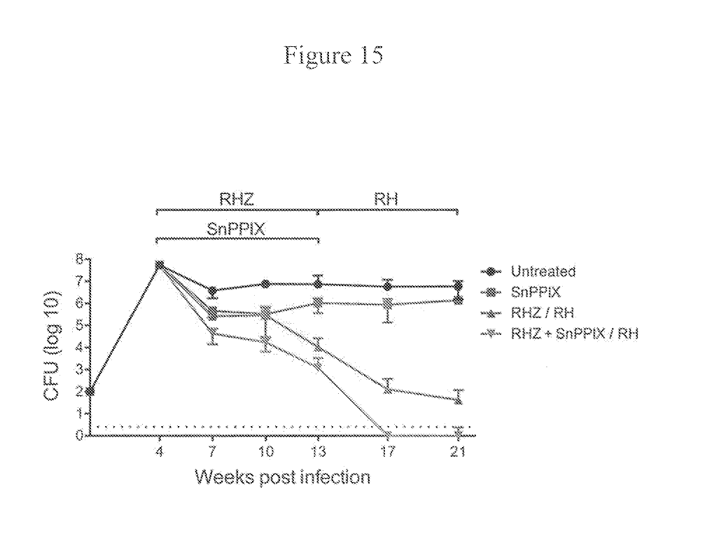

In a time course experiment it was found that the major additive effects of SnPPIX on RHZ treatment were observed during the first three weeks of drug administration. The combined therapy resulted in undetectable bacterial loads at 17 weeks after initiation of RHZ treatment, while in mice treated with RHZ alone bacteria were still detected as late as 21 weeks after initiation of treatment. The results are presented in FIG. 15.

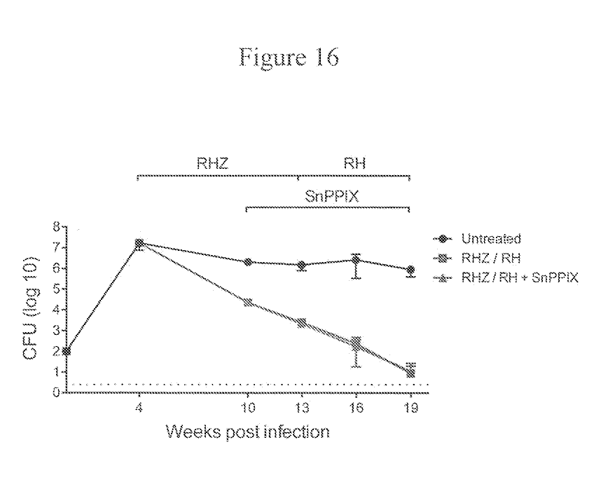

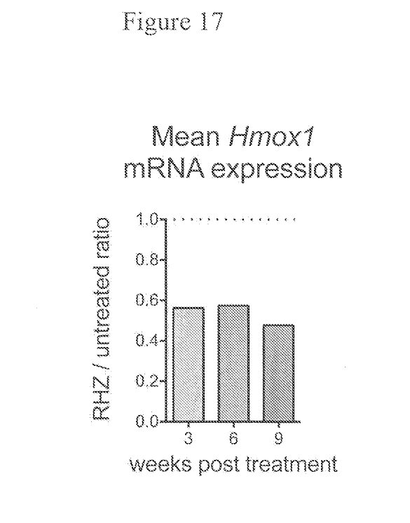

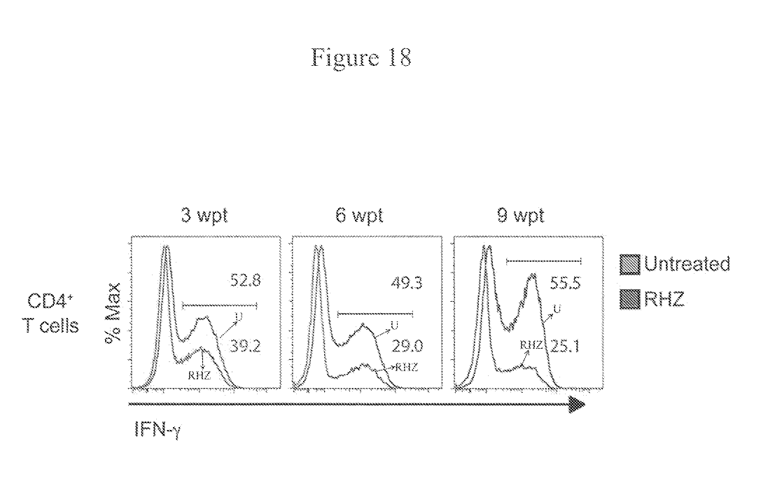

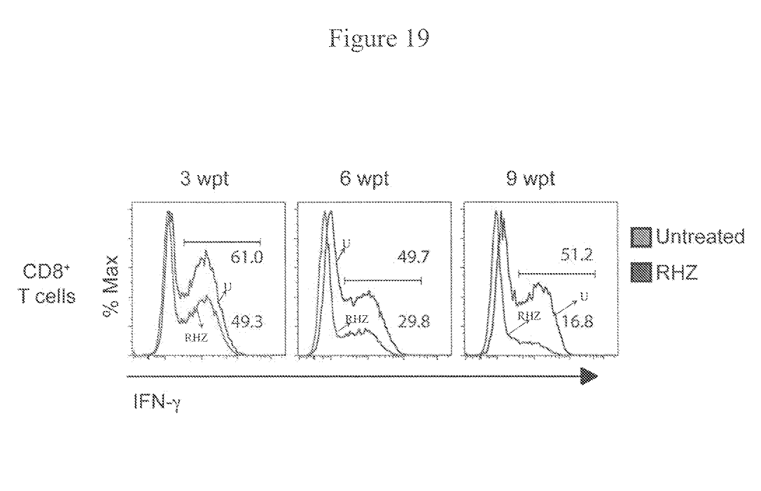

Although effective when administered at the same time as antibiotic treatment, SnPPIX supplementation failed to enhance RHZ efficacy when initiated 6 weeks after initiation of drug therapy. The results are presented in FIG. 16. This outcome may be due to a decline in host HO-1 expression following RHZ administration (see FIG. 17), which was temporarily correlated with both, the reduction in bacterial burden and the magnitude of the accompanying CD4 and CD8 T cell gamma interferon response at 3 weeks after initiation of RHZ therapy. The IFN-γ expression in CD4+ and CD8+T lymphocytes in lung homogenates of untreated or RIIZ treated Mtb-infected mice at the indicated time points after initiation of therapy are shown in FIG. 18 and FIG. 19, respectively.

It was also found that addition of SnPPIX to in vitro M. tuberculosis-infected macrophages resulted in reduced cellular bacterial load, and addition of FeSO₄ or FeCl₃ (which are iron-donating) were able to reverse this effect in a dose-dependent manner. It is known that excessive accumulation of heme, the substrate for HO-1, can induce production of reactive oxygen species (ROS), which are toxic for Mtb (Dutra F F et al., Front. Pharmacol., 5: 115 (2014); Akaki T et al., Clin. Exp. Immunol., 121:302-310 (2000)). Addition of SnPPIX to Mtb-infected bone marrow derived macrophages (BMDM) resulted in a decrease in the number of bacteria. However, addition of scavengers of ROS did not revert the HO-1 inhibition-driven reduction in bacterial load, suggesting that the aforementioned effect is not associated with ROS production. One of the products of heme degradation by HO-1 is labile iron, which is a key nutrient for Mtb, serving as a critical element in metabolic processes and bacterial survival. To determine if heme degradation by HO-1 could be serving as a source of iron at the intracellular compartment to be utilized by the pathogen, addition of FeSO₄ or FeCl₃, two iron donor compounds, were tested to see if they were capable of inhibiting the SnPPIX treatment-induced bacterial load reduction in Mtb-infected BMDM. Both molecules, when added alone to the infected macrophage cultures did not significantly enhance the number of bacteria, but when incubated in conjunction with SnPPIX, completely inhibited the reduction in bacillary load that occurs in response to HO-1 inhibition. Moreover, when Mtb-infected BMDM were incubated with 2′2′dipyridyl (DP) or pyridoxal isonicotinoyl hydrazone (PIH), two iron chelating agents, there was a significant reduction in the intracellular bacterial burden, and when these reagents were added to SnPPIX, a further enhancement in the reduction in the number of bacilli was observed. These results suggest that HO-1 may be working by generating intracellular labile iron to be used by the bacteria in Mtb-infected cells and, therefore, the upregulation in the production of the enzyme during TB may be detrimental to the host, by favoring bacterial survival and replication. Blocking of HO-1 activity through SnPPIX administration on the other hand, could favor Mtb killing by infected cells by decreasing iron release from heme degradation, resulting in decreased intracellular concentration of this essential key nutrient for bacterial survival.

Additional in vivo experiments have been conducted to characterize HO-1-expressing cells in the lungs of Mtb-infected mice. The results from these experiments indicated that after infection establishment, monocyte-derived myeloid cells are the major subset to express the enzyme at the organ. These cells accumulated at the lungs of wild type (WT) C57BL/6 mice only after 3 weeks post infection, and were also present at the lungs of T-cell deficient mice by this time point, however, at lower numbers as compared to WT mice. The precise mechanism through which T cells regulate HO-1 expression at the lungs of Mtb-infected mice is not yet known. It may be that cytokines, like TNF-α and IFN-γ, secreted by CD4+ T cells, which induce ROS production in phagocytes, can be indirectly triggering HO-1 production in Mtb-infected cells, once these metabolites trigger production of the enzyme. Another alternative is that chemokines produced by T lymphocytes or other cells in response to T cell-derived cytokines could be responsible for recruiting monocytic cells to the lungs of Mtb-infected mice. Monocytic cells were found to be the leukocyte population responsible for the majority of HO-1 expression in that organ.

Not wishing to be bound by any theory of mechanism, the following paragraph is provided. The modulation of the expression of other iron transporter proteins at the surface of myeloid leukocytes during Mtb infection, especially in HO-1-expressing cells was studied. In particular, it was observed that the expression of ferroportin, a protein that transports iron from the cytoplasm to the extracellular space, was downmodulated at the surface of HO-1+ pulmonary cells during Mtb infection. The expression of ferroportin on the surface of cells is subject to posttranslational regulation by a protein called hepcidin, which is produced by hepatocytes and macrophages and can bind to surface ferroportin, inducing its internalization and degradation. An increase in hepcidin serum levels at 15 days post Mtb infection was observed. This is the same time point at which ferroportin expression in HO-1+ cells starts to decrease. Therefore, Mtb infection results in upregulation of HO-1 expression, which catalyzes a reaction that releases iron in the cytoplasm, while, probably through induction of hepcidin production, it also induces down-modulation in the levels of ferroportin expression, which sends iron to the outside of the cell. Taken together these data strongly suggest that iron retention occurs in these cells during Mtb infection, a scenario that can favor bacterial survival and replication. While the data presented here indicates that blocking of HO-1 activity may be an efficient way to decrease iron accumulation in infected cells, consequently facilitating bacterial killing, interfering with the hepcidin-ferroportin axis, in a way to prevent ferroportin downmodulation in response to infection, may prove to be an even more effective host-directed therapy strategy, if employed in conjunction with HO-1 activity blockade.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Also, everywhere “comprising” (or its equivalent) is recited, the “comprising” is considered to incorporate “consisting essentially of” and “consisting of.” Thus, an embodiment “comprising” (an) element(s) supports embodiments “consisting essentially of” and “consisting of” the recited element(s). Everywhere “consisting essentially of” is recited is considered to incorporate “consisting of.” Thus, an embodiment “consisting essentially of” (an) element(s) supports embodiments “consisting of” the recited element(s). Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preventing or treating a Mycobacterium tuberculosis (Mtb) infection in a mammal, the method comprising:
administering to the mammal a first inhibitor, wherein the first inhibitor is an inhibitor of heme degradation and wherein the first inhibitor is a metal protoporphyrin, and
administering to the mammal a second inhibitor, wherein the second inhibitor is an inhibitor of Mtb,
wherein administration of the first and second inhibitors to the mammal prevents or treats Mtb infection in the mammal.

2. The method of claim 1, wherein the first inhibitor is tin (IV) protoporphyrin IX dichloride, zinc (II) protoporphyrin IX, gallium (III) protoporphyrin IX chloride, chromium (III) protoporphyrin IX choride, tin (IV) mesoporphyrin IX dichloride, chromium (III) mesoporphyrin IX choride, or zinc (II) deuteroporphyrin IX 2,3, bisethyleneglycol.

3. The method of claim 2, wherein the first inhibitor is tin (IV) protoporphyrin IX dichloride.

4. The method of claim 2, wherein the first inhibitor is zinc (II) protoporphyrin IX.

5. The method of claim 2, wherein the first inhibitor is tin (IV) mesoporphyrin IX dichloride.

6. The method of claim 1, wherein the second inhibitor is pyrazinamide, rifampicin, isoniazid, or any combination thereof.

7. The method of claim 1, wherein the first and second inhibitors are administered simultaneously.

8. The method of claim 1, wherein the first and second inhibitors are administered sequentially.

9. The method of claim 1, wherein the mammal is a human.

10. The method of claim 6, wherein the first inhibitor is tin (IV) protoporphyrin IX dichloride.

11. The method of claim 6, wherein the first inhibitor is zinc (II) protoporphyrin IX.

12. The method of claim 6, wherein the first inhibitor is tin (IV) mesoporphyrin IX dichloride.


SEQUENCE LISTING (7 sequences)

SEQ ID NO: 1 - DNA, 20 bp, Artificial Sequence
  synthetic
agctgcgttttacacccttt

SEQ ID NO: 2 - DNA, 20 bp, Artificial Sequence
  synthetic
aagccatgccaatgttgtct

SEQ ID NO: 3 - DNA, 20 bp, Artificial Sequence
  synthetic
gccaccaaggaggtacacat

SEQ ID NO: 4 - DNA, 20 bp, Artificial Sequence
  synthetic
gcttgttgcgctctatctcc

SEQ ID NO: 5 - DNA, 20 bp, Artificial Sequence
  synthetic
ttatgcagtcttgccggtcc

SEQ ID NO: 6 - DNA, 20 bp, Artificial Sequence
  synthetic
aacgctacttcgtggtgaca

SEQ ID NO: 7 - DNA, 20 bp, Artificial Sequence
  synthetic
cgtcaagcacgacctcgaat